(19) United States
(12) Patent Application Publication
(10) Pub. No.: US 2022/0257502 A1
(43) Pub. Date: Aug. 18, 2022

(54) CONTROLLED RELEASE FORMULATION DELIVERY DEVICE

(71) Applicant: INCUBE LABS, LLC, San Jose, CA (US)

(72) Inventor: Mir A. IMRAN, Los Altos Hills, CA (US)

(73) Assignee: INCUBE LABS, LLC, San Jose, CA (US)

(21) Appl. No.: 17/715,870

(22) Filed: Apr. 7, 2022

Related U.S. Application Data
(63) Continuation of application No. PCT/US2020/054550, filed on Oct. 7, 2020.
(60) Provisional application No. 62/912,581, filed on Oct. 8, 2019.

(51) Int. Cl. A61K 9/00; A61K 47/34; A61K 47/14; A61K 9/16; A61M 5/142

(52) U.S. Cl. CPC A61K 9/0004; A61K 9/0053; A61K 47/34; A61M 2005/14513; A61K 9/1647; A61K 9/0024; A61M 5/14276; A61K 47/14

(57) ABSTRACT

A desired elution profile can be identified, and a delivery device designed to effect the desired elution profile, such as by selecting materials for the constituent components of the delivery device, designing a structure of the delivery device and its constituent components, and selecting a content of formulations, to provide for the designed elution profile at particular expected conditions or at particular times or both.

### CROSS-REFERENCE TO RELATED APPLICATION

This application is a continuation of International Application No. PCT/US2020/054550 filed on Oct. 7, 2020, which claims the benefit of U.S. Provisional Patent Application No. 62/912,581, filed Oct. 8, 2019, which are incorporated herein by reference in their entireties.

### BACKGROUND

Many formulations are delivered by way of repeated multiple applications of amounts of the formulation, or by providing a treatment at one location with the intent for the formulation to be carried to another location by the natural processes of the environment. For example with respect to therapeutic treatment regimens, many therapeutic formulations are delivered by way of repeated injections (e.g., intramuscular, subcutaneous, or intravenous injections), which can be painful and/or inconvenient. Repeated injections also can distribute the formulation throughout a body rather than directly to an intended target location within the body, thereby exposing much more of the body to the formulation, and also requiring a higher dosage of the formulation at the injection site than is needed at the target location to account for losses in the body as the formulation travels to the target location.

It would be desirable to have a capability to deliver formulations in fewer applications and to deliver the formulations more directly to a target delivery site.

### SUMMARY

A desired elution profile can be identified, and a delivery device designed to effect the desired elution profile, such as by selecting materials for the constituent components of the delivery device, designing a structure of the delivery device and its constituent components, and selecting a content of formulations, to provide for the designed elution profile at particular expected conditions or at particular times or both.

### DRAWINGS

FIG. 1A, FIG. 1B, FIG. 1C, FIG. 1D, and FIG. 1E illustrate examples of shapes for embodiments of shells of a delivery device.

FIG. 2A, FIG. 2B, FIG. 2C, and FIG. 2D illustrate examples of shapes for embodiments of shells of a delivery device.

FIG. 3A, FIG. 3B, FIG. 3C, FIG. 3D, FIG. 3E, FIG. 3F, FIG. 3G, FIG. 3H, FIG. 3I, and FIG. 3J illustrate examples of shapes for embodiments of shells and orifices thereof of a delivery device.

FIG. 4A, FIG. 4B, FIG. 4C, and FIG. 4D illustrate examples of shapes for embodiments of plugs of a delivery device.

FIG. 5A, FIG. 5B, FIG. 5C, and FIG. 5D illustrate examples of shapes for embodiments of plugs of a delivery device.

FIG. 6A, FIG. 6B, and FIG. 6C illustrate examples of embodiments of a delivery device including multiple chambers.

FIG. 7A, FIG. 7B, FIG. 7C, and FIG. 7D illustrate examples of embodiments of a delivery device including multiple chambers and multiple plugs.

FIG. 8A and FIG. 8B illustrate examples of embodiments in which formulations are disposed in chambers of a delivery device.

FIG. 9 illustrates an example of an embodiment of a delivery device including multiple internal walls.

FIG. 10 illustrates an example of an embodiment of a delivery device including a chamber structure.

FIG. 11, FIG. 12, FIG. 13, FIG. 14, FIG. 15, FIG. 16, and FIG. 17 illustrate examples of embodiments of delivery devices.

FIG. 18 and FIG. 19 illustrate examples of embodiments of delivery devices having a pointed end defined by a plug.

FIG. 20 illustrates an example of an embodiment of a delivery device having a pointed end defined by a shell.

FIG. 21, FIG. 22, and FIG. 23 illustrate examples of embodiments of a delivery device having a pointed end including a tip component.

FIG. 24A, FIG. 24B, FIG. 24C, and FIG. 24D illustrate an example of a process for forming an embodiment of a delivery device including a tip component.

FIG. 25A and FIG. 25B illustrate an example of an embodiment of a delivery device including a puncture mechanism.

FIG. 26A and FIG. 26B illustrate examples of an embodiment of a delivery device including electronics.

FIG. 27A and FIG. 27B illustrate an example of an embodiment of a delivery device including an osmotic pump.

FIG. 28A and FIG. 28B illustrate an example of an embodiment of a delivery device including an osmotic pump.

FIG. 29A and FIG. 29B illustrate an example of an embodiment of a delivery device including an osmotic pump.

FIG. 30A and FIG. 30B illustrate an example of an embodiment of a delivery device including an osmotic pump.

FIG. 31A and FIG. 31B illustrate an example of an embodiment of a delivery device including an osmotic pump.

FIG. 32A and FIG. 32B illustrate an example of an embodiment of a delivery device including an osmotic pump.

FIG. 33A illustrates an example of an embodiment of a delivery device including orifices in two ends of the delivery device.

FIG. 33B, FIG. 33C, and FIG. 33D illustrate examples of embodiments of plugs such as could be used in the orifices of a delivery device, such as the delivery device illustrated in FIG. 33A.

FIG. 34 illustrates an example design of a delivery device, structured in accordance with concepts of the present disclosure.

FIG. 35 illustrates an example design of a delivery device, structured in accordance with concepts of the present disclosure.

FIG. 36 illustrates an example design of a delivery device including an osmotic pump, structured in accordance with concepts of the present disclosure.

FIG. 37 illustrates an example of an embodiment of a shell of a delivery device.

### DETAILED DESCRIPTION

When used in the present disclosure, the terms “e.g.”, “such as”, “for example”, “examples of”, and “by way of example” indicates that a list of one or more non-limiting example(s) precedes or follows; it is to be understood that other examples not listed are also within the scope of the present disclosure.

The term “biological matter” refers herein to blood, tissue, fluid, enzymes, and other secretions of the body. The term “digestive matter” refers herein to biological matter along the GI tract, and other matter (e.g., food in an undigested or a digested form) traversing the GI tract.

The terms “degrade”, “degrading”, “degraded”, and “degradation” refer herein to weakening, partially degrading, or fully degrading, such as by dissolution, chemical degradation, decomposition, chemical modification, mechanical degradation, or disintegration, which encompasses also, without limitation, dissolving, crumbling, deforming, or shrinking. The term “non-degradable” refers to an expectation that degradation will be minimal, or within a certain acceptable percentage, for an expected duration in an expected environment.

The term “degradation rate” (or “rate of degradation” or the like) refer herein to a rate at which a material degrades. A designed degradation rate of a material in a particular implementation can be defined by a rate at which the material is expected to degrade under expected conditions (e.g., in physiological conditions) at a target delivery location. A designed degradation time for an implementation can refer to a designed time to complete degradation or a designed time to a partial degradation sufficient to accomplish a design purpose (e.g., breach). Accordingly, a designed degradation time can be specific to a delivery device and/or specific to expected conditions at a target delivery location. A designed degradation time can be short or long and can be defined in terms of approximate times, maximum times, or minimum times. For example, the designed degradation time for a component can be about 1 minute, less than 1 minute, greater than 1 minute, about 5 minutes, less than 5 minutes, greater than 5 minutes, about 30 minutes, less than 30 minutes, greater than 30 minutes, and so forth with respect to minutes; or about 1 hour, less than 1 hour, greater than 1 hour, about 2 hours, less than 2 hours, greater than 2 hours, and so forth with respect to hours; or about 1 day, less than 1 day, greater than 1 day, about 1.5 days, less than 1.5 days, greater than 1.5 days, about 2 days, less than 2 days, greater than 2 days, and so forth with respect to days; or about 1 week, less than 1 week, greater than 1 week, about 2 weeks, less than 2 weeks, greater than 2 weeks, about 3 weeks, less than 3 weeks, greater than 3 weeks, and so forth with respect to weeks; or about 1 month, less than 1 month, greater than 1 month, about 2 months, less than 2 months, greater than 2 months, about 6 months, less than 6 months, greater than 6 months, and so forth with respect to months; or about 1 year, less than 1 year, greater than 1 year, about 2 years, less than 2 years, greater than 2 years, about 5 years, less than 5 years, greater than 5 years, about 10 years, less than 10 years, greater than 10 years, and so forth with respect to years; or other designed degradation approximate time, minimum time, or maximum time. A designed degradation time can be defined in terms of a limited range. For example, a designed degradation time can be in terms of a range of about 12-24 hours, about 1-6 months, about 1-2 years, or other range. Without wishing to be bound by any particular theory, controlled degradation can facilitate sustained and controlled release of payloads.

The terms “design”, “designing”, and “designed” refer herein to characteristics intentionally incorporated into a design based on estimates of tolerances related to the design (e.g., component tolerances and/or manufacturing tolerances) and estimates of environmental conditions expected to be encountered by the design (e.g., temperature, humidity, external or internal ambient pressure, external or internal mechanical pressure or stress, age of product, physiology, body chemistry, biological composition and/or chemical compositions of fluids and tissue, pH, species, diet, health, gender, age, ancestry, disease, tissue damage, or the combination of such); it is to be understood that actual tolerances and environmental conditions before and/or after ingestion can affect such designed characteristics so that different ingestible devices with a same design can have different actual values with respect to those designed characteristics. Use of the terms “design”, “designing”, and “designed” herein encompasses also variations or modifications to the design, a component structured (defined below) in accordance with the design, and design modifications implemented on a component after it is manufactured (defined below).

The term “fluid” refers herein to a gas or a liquid, or a combination thereof, and encompasses moisture and humidity. The term “fluidic environment” refers herein to an environment in which one or more fluids are present. In one or more embodiments, a delivery device in accordance with the present disclosure is structured to be disposed within a body, and thus biological matter or digestive matter results in a fluidic environment.

The terms “ingest”, “ingesting”, and “ingested” refer herein to taking into the stomach, whether by swallowing or by other means of depositing into the stomach (e.g., by depositing into the stomach by endoscope or depositing into the stomach via a port).

The terms “manufacture”, “manufacturing”, and “manufactured” as related to a component refer herein to making the component, whether made wholly or in part by hand or made wholly or in part in an automated fashion.

The term “structured” refers herein to a component or system that is manufactured according to a concept or design or variations thereof or modifications thereto (whether such variations or modifications occur before, during, or after manufacture) whether or not such concept or design is captured in a writing.

The terms “substantially” and “about” are used herein to describe and account for small variations. For example, when used in conjunction with a numerical value, the terms can refer to a variation in the value of less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

As used herein, a range of numbers includes any number within the range, or any sub-range if the minimum and maximum numbers in the sub-range fall within the range. Thus, for example, “<9” can refer to any number less than nine, or any sub-range of numbers where the minimum of the sub-range is greater than or equal to zero and the maximum of the sub-range is less than nine.

A delivery device as described herein delivers a payload to a site, such as a site within a body (e.g., a human or other animal body). A payload can be or include a formulation, electronics, another delivery device, or a combination of two or more of the foregoing.

In accordance with one or more embodiments of the present disclosure, a formulation can include one or more agents for delivery after administration or implantation of the delivery device. A formulation may be in a powder form or in a condensed or a consolidated form, such as a tablet or microtablet. A delivery device can include one or more formulations. A wide range of agents can be used. For example, agents can be, or can include, any pharmacologically active agent (e.g., antibiotic, NSAID, angiogenesis inhibitor, neuroprotective agent, chemotherapeutic agent), a DNA or SiRNA transcript (e.g., for modifying genetic abnormalities, conditions, or disorders), a cell (e.g., produced by or from living organisms or contain components of living organisms), a cytotoxic agent, a diagnostic agent (e.g., sensing agent, contrast agent, radionuclide, fluorescent moiety, luminescent moiety, magnetic moiety), a prophylactic agent (e.g., vaccine), a nutraceutical agent (e.g., vitamin, mineral, herbal supplement), a delivery enhancing agent, a delay agent, an excipient, a substance for cosmetic enhancement, another substance, or any combination of two or more of the foregoing. In delivery sites within a body, an agent can be suitable for introduction to biological tissues.

Examples of pharmacologically active agents include peptides, proteins, immunoglobulins (e.g., antibodies), large molecules, small molecules, hormones, and biologically active variants and derivatives of any of the foregoing.

An agent can be in a class of antibodies, such as immunoglobulin G (e.g., a TNF-alpha antibody such as adalimumab), an interleukin in the IL-17 family of interleukins (e.g., brodalumab, secukinumab, ixekizumab), an anti-eosinophil antibody, or any other class, and can be humanized or not.

Examples of nutraceutical agents include vitamin A, thiamin, niacin, riboflavin, vitamin B-6, vitamin B-12, other B-vitamins, vitamin C (ascorbic acid), vitamin D, vitamin E, folic acid, phosphorous, iron, calcium, and magnesium.

Examples of cells include stem cells, red blood cells, white blood cells, neurons, and other viable cells.

Examples of vaccines include vaccines against various bacteria and viruses or proteins thereof (e.g., influenza, meningitis, human papillomavirus (HPV), or chicken pox). In various embodiments of vaccines to viruses, the vaccine can correspond to various attenuated viruses.

Examples of delivery enhancement agents include a permeation enhancer, an enzyme blocker, a peptide that permeates through mucosa, an antiviral drug such as a protease inhibitor, a disintegrant or superdisintegrant, or a pH modifier. A delivery enhancing agent can, for example, serve as a delivery medium for delivery of one or more agents (e.g., therapeutic agents) or serve to improve absorption of one or more agents into the body. In one or more embodiments, a delivery enhancing agent primes an epithelium of the intestine (e.g., fluidizes an outer layer of cells) to improve absorption and/or bioavailability of one or more other agents included in the delivery device.

Delivery enhancing agents include, for example, surfactants, bile salts, fatty acids, chelating agents, chitosans, and derivatives of any of the foregoing. Specific examples of delivery enhancing agents include sodium lauryl sulphate, sodium dodecylsulphate, dioctyl sodium sulfosuccinate, polysorbitate, sodium glycholate, sodium deoxycholate, sodium taurocholate, sodium dihydrofusidate, sodium glycodihdro fusidate, oleic acid, caprylic acid, lauric acid, nonylphenoxypolyoxetyylene, TWEEN® 80, medium chain fatty acid-based sodium caprate, sodium caprylate, 8-(N-2-hydroxy-5-chloro-benzoyl)-amino-caprylic acid (5-CNAC), sodium N-[8-(2-hydroxylbenzoyl)amino]caprylate (SNAC), omega 3 fatty acid acylcarnitine, acylcholine, ethylenediaminetetraacetic acid (EDTA), citric acid, salicylate, N-sulfanto-N,O-carboxymethylchitosan, N-trimethylated chloride, chitosan glutamate, alkylglycoside, lipid polymer, zonula occludens toxin, polycarbophyl-cystein conjugate, and a derivative of any of the foregoing.

In one or more embodiments, a formulation can include one or more vasodilation agents (e.g., l-arginine, Sildenafil, nitrate (e.g., nitroglycerin), epinephrine), or a vasoconstrictor (e.g., stimulants, amphetamines, antihistamines, epinephrine, cocaine).

A formulation can also include one or more excipients to provide an appropriate medium for one or more agents included in an embodiment of the formulation (e.g., for assisting in manufacture), or to preserve integrity of one or more agents included in the formulation (e.g., during manufacture, during storage, or after ingestion prior to dispersion within the body).

Examples of excipients include binders, disintegrants and superdisintegrants, buffering agents, anti-oxidants, and preservatives.

A delay agent can be included with (e.g., mixed with, or providing a structure around) one or more other agent(s) in a formulation to slow a release rate of the other agent(s) from the formulation. Examples of delay agents include poly(lactic acid) (PLA), poly(glycolic acid) (PGA), polyethylene glycol (PEG), poly(ethylene oxide) (PEO), poly (l-lactic acid) (PLLA), poly(D-lactic acid) (PDLA), other polymers, hydrogel, and combinations of two or more of the foregoing.

As noted above, each formulation can include one or more agents, and a delivery device can include one or more formulations. Accordingly, an embodiment of the delivery device can include one agent or multiple agents.

Embodiments of delivery devices in accordance with the present disclosure are structured to provide the payload in accordance with a predefined delivery timeline and profile. Such timeline can be in terms of seconds, minutes, hours, days, weeks, months, or years.

Having described a delivery device in overview, various embodiments will next be discussed with reference to the figures.

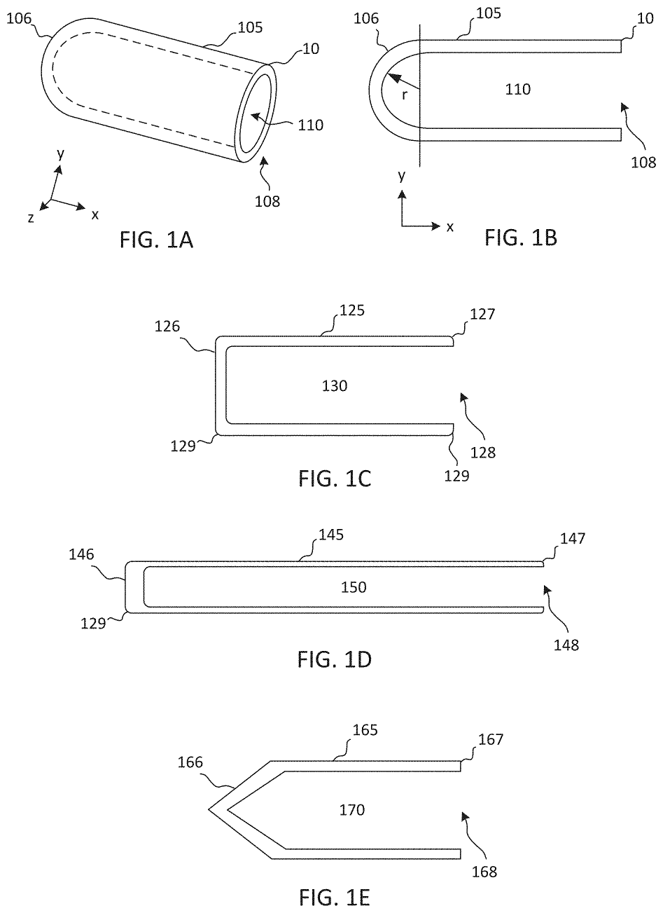

FIGS. 1A-1E illustrate examples of shapes for embodiments of shells of a delivery device. FIG. 1A represents a perspective view of a shell 105 in an x-y-z domain, and FIG. 1B represents a slice view of shell 105 in an x-y plane of the x-y-z domain at z=0 (i.e., along a central axis). Shell 105 is approximately cylindrical, having a closed end 106 with a hemispherical or semi-hemispherical shape having an inner radius ‘r’. Another end 107 defines an orifice 108, which in this example is fully open, extending across the entirety of end 107. Shell 105 defines a cavity 110 in communication with orifice 108.

FIG. 1C and FIG. 1D illustrate additional examples of shapes for embodiments of shells of a delivery device, shown in slice view in an x-y plane of the x-y-z domain at a point in a z direction. In FIG. 1C and FIG. 1D, a closed end of the shell is substantially flat (approximately no curvature) as compared to end 106 of shell 105 in FIGS. 1A, 1B. In FIG. 1C, a shell 125 has an end 126 which is substantially flat, and an end 127 which defines a fully open orifice 128 in communication with a cavity 130 defined by shell 125. In FIG. 1D, a shell 145 has an end 146 which is substantially flat, and an end 147 which defines a fully open orifice 148 in communication with a cavity 150 defined by shell 145. By way of comparison, shell 145 is longer and narrower than shell 125, illustrating that various absolute and relative shell dimensions are within the scope of the present disclosure. For example, a shell can be designed for its fit at a target delivery location, and/or designed for ease of delivery at a target location, and/or designed for convenience of manufacture. In FIG. 1C and FIG. 1D, corners (e.g., corners 129) are shown slightly rounded, illustrating that corner design, radiusing and/or manufacturing can result in a variety of corner shapes for any shell, including shell 125 or shell 145.

FIG. 1E illustrates another example of a shape for embodiments of shells of a delivery device, shown in slice view in an x-y plane of the x-y-z domain at a point in a z direction. In the example of FIG. 1E, a shell 165 has an end 166 which comes to a point somewhere on end 166. The pointed end 166 can define a cone when viewed in three dimensions, or can define another shape (e.g., the shape of a tip of a quill tip pen). In alternative embodiments, a portion of end 166 has a pointed shape and a remainder of end 166 has a different shape (e.g., a needle shape portion on a cylindrical or polygonal shaped base portion of end 166). Shell 165 has an end 167 defining a fully open orifice 168 in communication with a cavity 170 defined by shell 165.

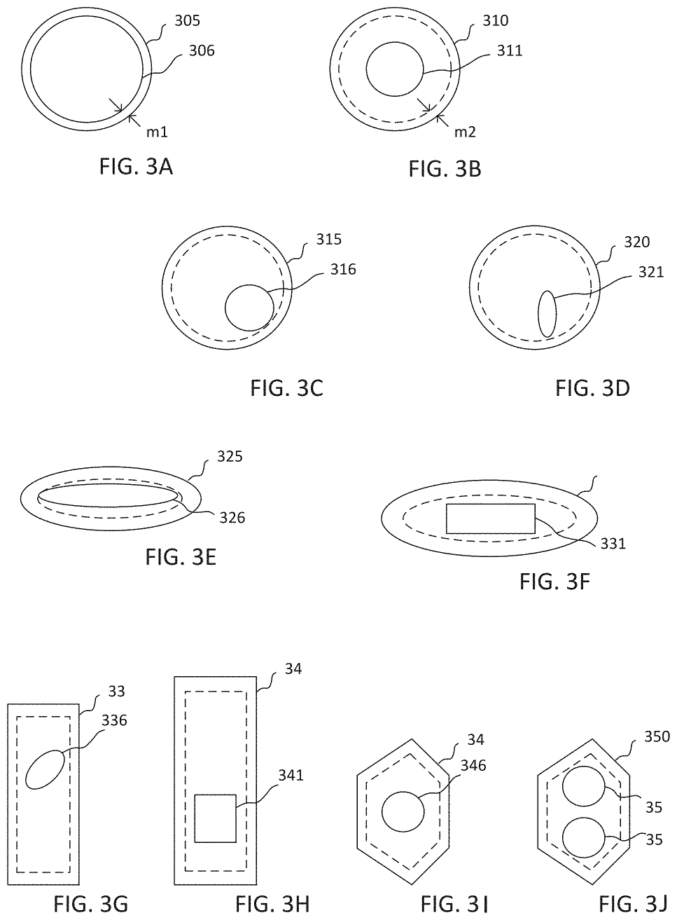

In FIGS. 1A-1E, each of shells 105, 125, 145, 165 has a fully open end (respectively end 107, 127, 147, 167) such that a size of an orifice (respectively orifice 108, 128, 148, 168) is defined by a material thickness of a wall of the respective shell across the respective end; in other embodiments, the orifice defined by the shell end does not extend across the entirety of the shell end (see, e.g., the discussion of FIG. 3A as compared to the discussion of FIG. 3B).

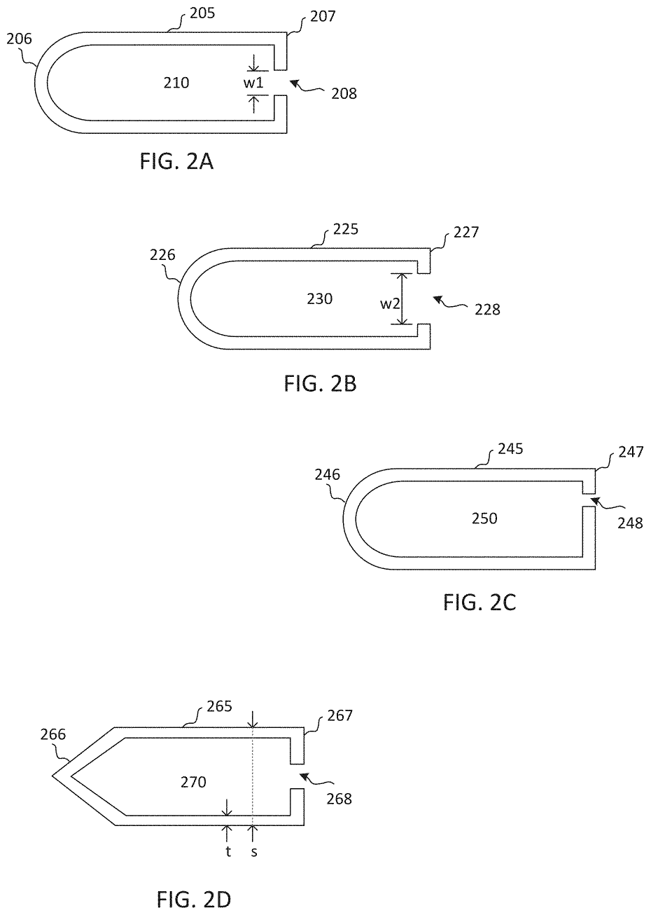

FIGS. 2A-2C illustrate examples of shells similar to shell 105 in FIGS. 1A, 1B, except for having partially closed ends. Referring to FIG. 2A, a shell 205 has a closed end 206, and an end 207 defining an orifice 208 in communication with a cavity 210 defined by shell 205. Orifice 208 extends through a portion of end 207. Referring to FIG. 2B, similar to FIG. 2A, a shell 225 has a closed end 226, and an end 227 defining an orifice 228 in communication with a cavity 230 defined by shell 225, where orifice 228 extends through a portion of end 227. Orifice 228 of FIG. 2B has a greater orifice dimension ‘w2’ as compared to a dimension ‘w1’ of orifice 208 of FIG. 2A, illustrating that an orifice can be designed with a desired dimension. Referring to FIG. 2C, similar to FIG. 2A, a shell 245 has a closed end 246, and an end 247 defining an orifice 248 in communication with a cavity 250 defined by shell 245, where orifice 248 extends through a portion of end 247. Orifice 248 of FIG. 2C is offset from a lengthwise centerline of shell 245, illustrating that an orifice can be designed at a desired position on a shell.

FIG. 2D illustrates an example of a shell 265 similar to shell 165 in FIG. 1E, except for having a partially closed end 267 defining an orifice 268 in communication with a cavity 270 defined by shell 265, rather than the fully open orifice 168 of shell 165 in FIG. 1E, illustrating that an orifice can be designed to have a desired size for any shape of shell.

In one or more embodiments, a shell (e.g., any of shell 105, 125, 145, 165, 205, 225, 245, or 265, or other shell embodiment) is constructed integrally, meaning that the entire shell is formed as a unit. In one or more other embodiments, a shell (e.g., any of shell 105, 125, 145, 165, 205, 225, 245, or 265, or other shell embodiment) is constructed using two or more components which are then assembled together to form the finished shell; in such embodiments, the components can be attached to each other in a semi-permanent or non-permanent structure with connection mechanisms (e.g., using snap features, hook-and-loop features, adhesives, adhesive tape) or attached to each other in a more permanent fashion (e.g., using heat staking, vibration welding, compression welding), or a combination thereof. An example of a shell incorporating multiple components is a tube cut to a desired length with a closed component attached at one end and a component with an orifice attached at the other end. Another example of a shell incorporating multiple components is a shell (e.g., any of shell 105, 125, 145, 165, 205, 225, 245, or 265, or other shell embodiment) molded in two halves lengthwise and attached together after molding.

In FIGS. 1A-1E and FIGS. 2A-2D, a thickness of material (e.g., thickness ‘t’ in FIG. 2D) of each of shells 105, 125, 145, 165, 205, 225, 245, 265 is illustrated as being consistent throughout the shell, within manufacturing tolerances. In other embodiments, portions of a shell can have a greater thickness, such as for wall strength in a particular portion, or such as wall strength generally (e.g., by using a corrugated or convoluted pattern), or such as to control a rate of delivery of a therapeutic agent. In one or more embodiments, a material thickness of an end of a shell (e.g., end 106, 126, 146, 166, 206, 226, 246, 266) is much thicker than at other portions of the shell, and thus a cavity of a shell of a delivery device (e.g., respective cavity 110, 130, 150, 170, 210, 230, 250, 270) may not extend into that end of the shell, or may not extend as far into the end of the shell as illustrated in the respective figures.

Although the shells shown in FIGS. 1A-1E (shells 105, 125, 145, 165) and FIGS. 2A-2D (shells 205, 225, 245, 265) are illustrated as having approximately uniform cross-sectional dimensions (e.g., dimension ‘s’ in FIG. 2D), other embodiments of shells have non-uniform cross-sectional dimensions. For example, a shell can be wider at a point near a lengthwise (x-axis) middle of the shell than at a point towards one of the ends of the shell. For another example, a point towards one of the ends of the shell can be wider than other points along the shell.

Additionally, cross-sectional shapes can vary along a length of a shell, or can be consistent along portions of, or the entirety of, the length of the shell. FIG. 3A-FIG. 3J provide a few examples of cross-sectional shapes.

FIG. 3A illustrates a view of an example shell 305 (e.g., an embodiment of shell 105 of FIG. 1A) as viewed towards an end of shell 305. Shell 305 defines an orifice 306 at this end, which extends fully across shell 305. This end of shell 305 is substantially circular, and orifice 306 is thus substantially circular, being defined by a material thickness ‘m1’ at this end of shell 305.

FIG. 3B illustrates an example shell 310 as viewed towards an end of shell 310. Shell 310 is similar to shell 305 of FIG. 3A except that an orifice 311 defined by this end of shell 310 extends partially across shell 310 as compared to orifice 306 in FIG. 3A that extends fully across shell 305. In FIG. 3B, the dotted line illustrates a material thickness ‘m2’ of shell 310, which does not define the size or shape of orifice 311 in this example.

FIG. 3C illustrates an example shell 315 as viewed towards an end of shell 315. Shell 315 is similar to shell 310 of FIG. 3B except that an orifice 316 defined by this end of shell 315 is offset from a centerline of shell 315 (e.g., offset from the x-axis) as compared to the orifice 311 in FIG. 3B that is approximately centered with respect to an outer circumference of that end of shell 310.

FIG. 3D illustrates an example shell 320 as viewed towards an end of shell 320. Shell 320 is similar to shell 315 of FIG. 3C except that an orifice 321 defined by this end of shell 320 has an elliptical shape rather than the circular shape of orifice 316 in FIG. 3C.

FIG. 3E illustrates an example shell 325 as viewed towards an end of shell 325. Shell 325 is similar to shell 320 of FIG. 3D except that this end of shell 325 has an elliptical shape rather than the circular shape of the end of shell 320 as illustrated in FIG. 3D. Further, shell 325 defines an elliptically-shaped orifice 326 at this end; orifice 326 extends substantially across shell 325 along the long axis of the ellipse and a portion of the way across shell 325 along the short axis of the ellipse.

FIG. 3F illustrates an example shell 330 as viewed towards an end of shell 330. Shell 330 is similar to shell 325 of FIG. 3E except that shell 330 defines a polygonal-shaped (here, rectangular-shaped) orifice 331 rather than the elliptical shape of the end of shell 325 as illustrated in FIG. 3E. Further, orifice 331 does not extend fully across this end of shell 330 in any direction.

FIGS. 3G and 3H illustrate polygon-shaped (in particular, rectangular-shaped) shells as viewed towards ends of the shells. A shell 335 in FIG. 3G defines an elliptically-shaped orifice 336 offset from a center of this end of shell 335, and rotated such that the long axis of the ellipse does not align with a long or short axis of this end of shell 335. A shell 340 in FIG. 3H defines a polygon-shaped (here, square-shaped) orifice 341 offset from a center of this end of shell 340.

FIGS. 3I and 3J illustrate additional polygon-shaped shells as viewed towards ends of the shells. FIG. 3I illustrates a polygon-shaped shell 345 defining a circular orifice 346 approximately centered at this end of shell 345. FIG. 3J illustrates a polygon-shaped shell 350 defining two circular orifices 351, 352. Multiple orifices in a shell (e.g., orifices 351, 352 in shell 350) can allow, for example, diffusion of a formulation from two orifices in communication with a cavity defined by the shell, or diffusion from two chambers of a shell as discussed below, or graduated diffusion in steps as discussed below, or for other diffusion profiles. The terms “diffuse”, “diffusion”, and “diffusing” as used herein indicates a movement across a space, along a surface, or through an orifice, and encompasses slow or fast movement (e.g., encompasses oozing, trickling, exuding, dripping, dribbling, flowing, discharging, excreting, leaking, draining, sweating, leaching, percolating, seeping, squirting, spurting, jetting, spraying, gushing, pouring, erupting, expelling, and other issuances).

Multiple orifices in a shell can additionally or alternatively provide for ease of manufacture, and/or to increase a strength of the shell by designing a total desired cross-sectional orifice area over multiple orifices.

FIGS. 3A-3J illustrate cross-sectional shapes as viewed towards an open end of a shell. In one or more embodiments, such cross-sectional shape (e.g., in a y-z plane) is consistent along a length (e.g., along an x-axis) of the shell. In one or more other embodiments, a cross-sectional shape (e.g., in a y-z plane) of a shell varies along its length (e.g., along an x-axis).

All components of a delivery device can be designed to be implemented for applications in which a target environment is biological (e.g., human or other animal). Accordingly, components of a delivery device (including the shell) can be designed for implementation using biocompatible materials, and in some embodiments, the materials are also degradable, and can further be biodegradable. Such materials include polymers (e.g., PLA, PGA, poly(lactic-co-glycolic acid) (PLGA), PLLA, polyglycolic acid-co-l-lactic acid (PGLA), PEG, polycaprolactone (PCL), a copolymer of any of the foregoing polymers such as dipolymer PLGA-PEG, or tripolymer PLGA-PEG-PLGA or PEG-PLGA-PEG, a combination of any of the foregoing polymers with another material or materials, a combination of any two or more of the foregoing), metals (e.g., magnesium (Mg), iron (Fe), tungsten (W), zinc (Zn), yttrium (Y), neodymium (Nd), zirconium (Zr), palladium (Pd), manganese (Mn), a combination of any of the foregoing metals with another material or materials, an alloy of any the foregoing metals, a combination of two or more of any of the foregoing), metallic glasses (e.g., those based on strontium (Sr) or calcium (Ca)), starch, sugar, other biodegradable materials, or any combination of two or more of the foregoing. Such materials further include hydrogels (hydrophilic polymer chains). The biodegradable materials can be selected based on desired properties for the particular component of the delivery device, such as rate of degradation, shear strength prior to or during degradation, brittleness, tensile strength, durability, bendability, manufacturability of the component incorporating the biodegradable material(s), compatibility with other materials used in the component, material stability (e.g., shelf life), temperature constraints, acidity constraints, and so forth.

In one or more embodiments, a shell of a delivery device is formed of PGA; in one or more embodiments, a shell of a delivery device is formed of PLA. PGA and PLA have different degradation rates. In one or more embodiments, a shell of a delivery device is formed of a PLA-PGA blend to select a different degradation time as compared to PGA or PLA alone. Other degradation times can be achieved by using different materials, in addition or alternatively.

Embodiments of the delivery device of the present disclosure provide for extended diffusion of a formulation from the delivery device. With respect to many embodiments of delivery devices according to the present disclosure, the delivery device is deployed to a target location where the target location is a fluidic environment. In a fluidic environment, if fluid enters the delivery device and encounters a degradable formulation, a process (e.g., an elution process or other degradation process, referred to generally herein as an elution process for convenience, disregarding the mechanism of the degradation) can begin whereby the formulation degrades in the presence of fluid and results in a fluidized combination of fluid and formulation (e.g., a chemical combination, or a slurry of particles in a fluidic carrier), referred to herein as a fluidized formulation.

When the elution process begins and the fluidized formulation begins to diffuse from the delivery device, there will be a higher concentration of fluidized formulation within the delivery device than external to the delivery device, until the elution process is completed and much or all of the fluidized formulation has diffused into fluid of the environment. A profile of diffusion of the fluidized formulation out of the delivery device by way of an elution process (alone or in combination with other processes) is referred to herein for convenience as an elution profile. A delivery device can be designed to provide for a predefined elution profile of the formulation or of an agent in the formulation. The predefined elution profile can be a general profile, or can be designed for specific environmental conditions (e.g., expected temperature, pH, humidity, motion, etc., or expected presence of particular fluid types, proteins or other molecules, and/or concentrations thereof).

Design of a delivery device to provide a given elution profile includes without limitation: selection of materials of component portions of the shell; design of shell size and shape; design of shell cavity size and shape; design of shell orifice(s) size, shape, and position; and, as applicable, design of a plug or cover (either of which is referred to hereinafter as a plug for ease of discussion) to fill, cover, or block an orifice or otherwise partially or fully prevent or minimize passage of materials (e.g., fluid, semi-solid material, particulates) through the orifice. A plug can be degradable or non-degradable.

In one or more embodiments, a plug can be attached to a shell (e.g., by heat stake, compression, adhesive). In one or more embodiments, a plug can be disposed in an orifice of a shell.

In one or more embodiments, a plug can be in a form to permit passage of certain materials while minimizing or preventing passage of other materials. Examples of materials that can be used in such plugs are meshing and membranes.

In one or more embodiments, a plug can permit passage of certain materials in one direction while minimizing or preventing passage of such materials in the opposite direction, such as a membrane.

In one or more embodiments, a degradable plug can provide for a time delay when desired for a particular elution profile. Material selection, thickness, and shape of such a plug defines the manner and rate at which the plug degrades in a given environment.

In one or more embodiments, a degradable plug or a non-degradable plug can be used in an orifice of the shell. In one or more embodiments, a combination of degradable and non-degradable plugs can be used at the same orifice of a shell. In one or more embodiments, a plug can be formed of both degradable and non-degradable materials, to provide for different plug characteristics at different times of the elution profile.

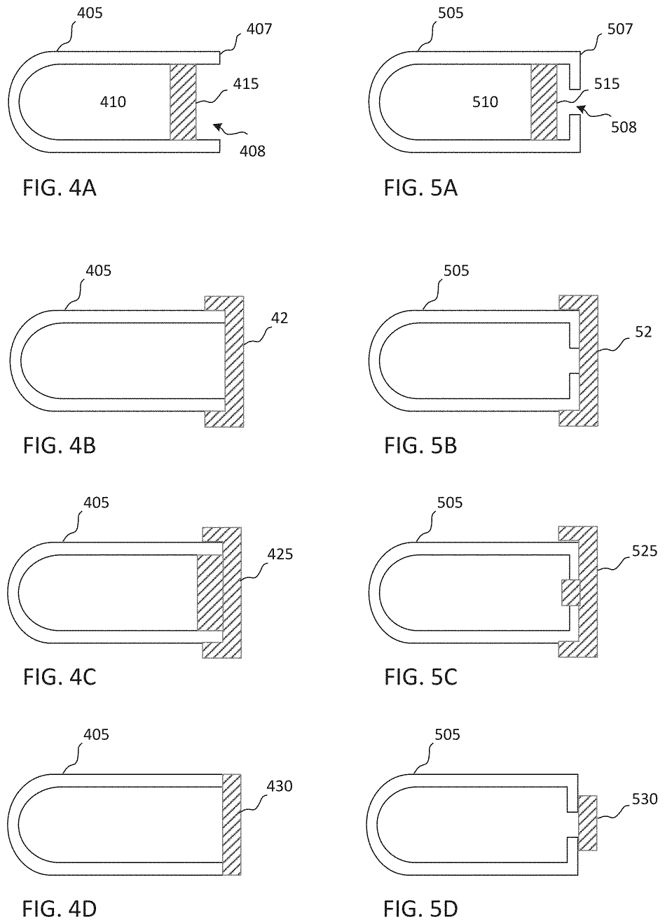

FIGS. 4A-4D illustrate examples of embodiments of plugs for an orifice 408 which extends fully across an end 407 of a shell 405 (similarly to orifice 108 of shell 105 in FIG. 1A). FIG. 4A illustrates a plug 415 disposed in a cavity 410 defined by shell 405. FIG. 4B illustrates a plug 420 disposed over end 407 of shell 405 and wrapping around an outside of shell 405, such as to hold plug 420 in place (e.g., by compression force, by snap fit over a lip of shell 405) or to extend a barrier formed by plug 420. FIG. 4C illustrates a plug 425 disposed partially within cavity 410 and partially exterior to shell 405. FIG. 4D illustrates a plug 430 disposed fully exterior to shell 405 but not designed to wrap around shell 405 as does plug 420 illustrated in FIG. 4B or plug 425 illustrated in FIG. 4C.

FIGS. 5A-5D illustrate examples of embodiments of plugs for an orifice 508 which extends partially across an end 507 of a shell 505 (similarly to orifice 208 of shell 205 in FIG. 2A). FIG. 5A illustrates a plug 515 disposed in a cavity 510 defined by shell 505. FIG. 5B illustrates a plug 520 disposed over end 507 of shell 505 and wrapping around an outside of shell 505. FIG. 5C illustrates a plug 525 disposed partially within cavity 510 and partially exterior to shell 505. FIG. 5D illustrates a plug 530 disposed fully exterior to shell 505 but not designed to wrap around shell 505 as does plug 520 illustrated in FIG. 5B or plug 525 illustrated in FIG. 5C.

An adhesive material (not shown) can be used to attach any of plugs 415, 420, 425, 430 to shell 405 or any of plugs 515, 520, 525, 530 to shell 505. Alternatively or additionally, any of plugs 415, 420, 425, 430, 515, 520, 525, 530 can be formed of a material which, when pressed against shell 405 or 505 (as applicable), conforms to or adheres to shell 405 or 505 so as to stay attached to shell 405 or 505 for a designed time period.

A plug (e.g., 415, 420, 425, 430, 515, 520, 525, 530) can be integrally formed or can be formed in multiple layers. The plug, or one or more layers thereof, can include one or more degradable materials. Thus, for example, although plug 425 in FIG. 4C and plug 525 in FIG. 5C are illustrated as having two layers each, either or both of plugs 425, 525 can be integrally formed.

A plug can be designed to be implemented in a biological environment. Accordingly, a plug can be designed for implementation using biocompatible materials, and in some embodiments, the materials can also be degradable, and can further be biodegradable. Such materials include polymers (e.g., PLA, PGA, PLGA, PLLA, PGLA, PEG, PCL, a combination of any of the foregoing polymers with another material or materials, a combination of any two or more of the foregoing), metals (e.g., Mg, Fe, W, Zn, Y, Nd, Zr, Pd, Mn, a combination of any of the foregoing metals with another material or materials, an alloy of any the foregoing metals, a combination of two or more of any of the foregoing), metallic glasses (e.g., those based on Sr or Ca), starch, sugar, other biodegradable materials, or any combination of two or more of the foregoing. Such materials further include hydrogels (hydrophilic polymer chains). The biodegradable materials can be selected based on desired properties for the particular plug, such as rate of degradation, shear strength prior to or during degradation, brittleness, tensile strength, durability, bendability, manufacturability of the plug incorporating the biodegradable material(s), compatibility with other materials used in the plug (or in other components of the delivery device), material stability (e.g., shelf life), temperature constraints, acidity constraints, and so forth.

In one or more embodiments, a plug of a delivery device is formed of PGA; in one or more embodiments, a plug of a delivery device is formed of PLA. PGA and PLA have different degradation rates. In one or more embodiments, a plug of a delivery device is formed of a PLA-PGA blend to select a different degradation time as compared to PGA or PLA alone. Other degradation times can be achieved by using different materials, in addition or alternatively. For example, a plug can include PLA and Mg.

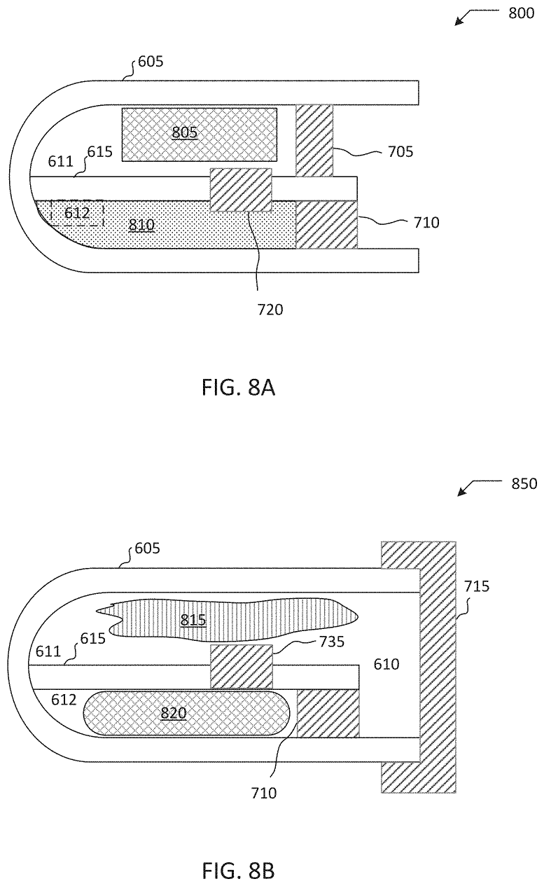

As introduced above with respect to the description of FIG. 3J, a delivery device can contain multiple chambers, meaning that a cavity of the shell of the delivery device is separated into different chambers. Separation into chambers can be, for example, by molding multiple chambers into the shell, or by adding structures within the shell to divide the cavity. The chambers can be, but are not necessarily, fully isolated from each other; in one or more embodiments, the chambers are not fully isolated from each other, meaning that material in one chamber can be allowed to flow to a neighboring chamber. In one or more embodiments, a wall between two chambers can be designed to define an orifice to allow a controlled dispersal of material unidirectionally from one of the chambers to the other, or bidirectionally between the chambers. Such an implementation can be useful, for example, to provide for a controlled mixing of materials, or to provide a controlled release of materials to a chamber in communication with an orifice to an exterior of the delivery device, or other purpose. In one or more embodiments, an orifice between two chambers is blocked by a plug (e.g., similar to one of the plugs described with respect to FIGS. 4A-4D, 5A-5D); the plug can be composed of degradable material(s), non-degradable material(s), or a combination of degradable and non-degradable materials. A non-degradable plug can be used between chambers of a shell, for example, to allow for use of a single shell design in multiple configurations (e.g., a configuration in which the chambers are fully separated versus a configuration in which the chambers are open to each other or become open to each other after degradation of a plug in the orifice therebetween). A degradable plug can be used between chambers, for example, to add a design delay with respect to implementing a predefined elution profile (see, e.g., FIG. 7C, FIG. 7D, FIG. 8A, FIG. 8B and the descriptions thereof).

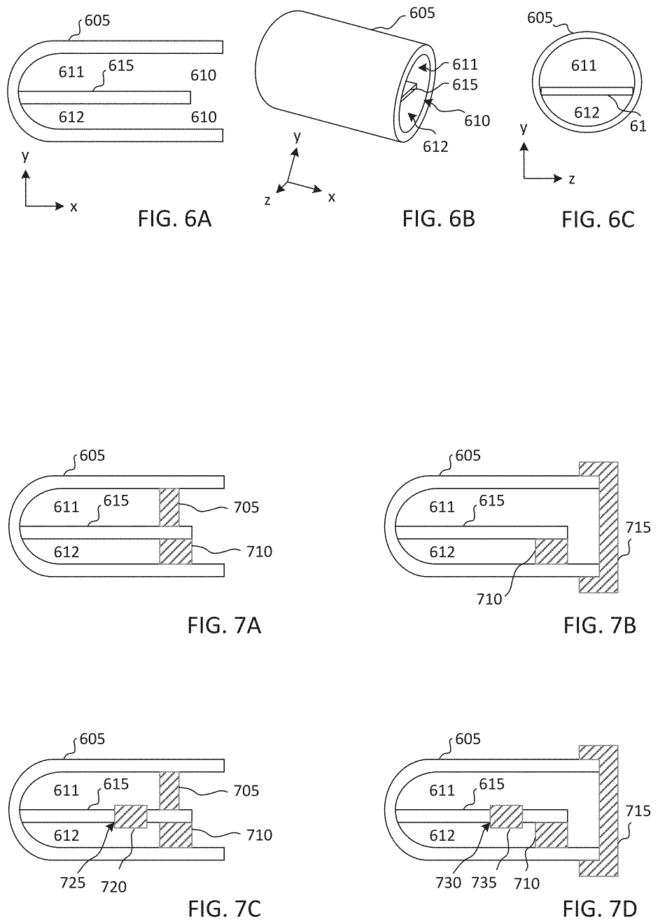

FIG. 6A illustrates in cross-sectional view an example of a shell 605 similar to shell 105 in FIG. 1A except that shell 605 defines a cavity 610 divided into two chambers 611, 612 by a wall 615 disposed in cavity 610 (e.g., integral with, or positioned within, shell 605). FIG. 6B illustrates an embodiment of shell 605 in perspective view showing that wall 615 extends within cavity 610 to form chambers 611, 612. FIG. 6C illustrates shell 605 as seen facing an open end of an embodiment of shell 605. The examples of wall 615 and chambers 611, 612 are provided by way of illustration with respect to shell 605, and are applicable also to other shell designs (e.g., shell 125, 145, 165, 205, 225, 245, 265, or other shell). Further, other positions and shapes of walls, and thus other shapes and relative sizes of chambers, are within the scope of the present disclosure, and multiple walls can define three or more chambers.

FIG. 7A illustrates an example of shell 605 incorporating a plug 705 to block chamber 611 and a plug 710 to block chamber 612. It is to be understood that plugs 705, 710 are not limited to the design illustrated, and can take any of many different forms applicable to the design of shell 605 and wall 615. Further, additional or alternative plugs can be used with respect to shell 605, such as one or more of the plugs illustrated in FIGS. 4A-4D.

FIG. 7B illustrates an example of shell 605 in the configuration of FIG. 7A with plug 705 omitted and a plug 715 added to block cavity 610.

One or more plugs can be used to block flow of material through any wall of a chamber.

FIG. 7C illustrates an example of shell 605 in the configuration of FIG. 7A with an additional plug 720 disposed in an orifice 725 defined by wall 615 between chambers 611, 612.

FIG. 7D illustrates an example of shell 605 in the configuration of FIG. 7B with an additional plug 735 disposed in an orifice 730 defined by wall 615 between chambers 611, 612.

To illustrate how a delivery device according to an embodiment of the present disclosure can be designed to implement a desired elution profile, examples will next be described with respect to FIGS. 8A, 8B.

FIG. 8A illustrates a delivery device 800 including the shell 605 configuration of FIG. 7C, with a first formulation 805 disposed in chamber 611 and a second formulation 810 disposed in chamber 612.

In a first example with respect to delivery device 800 in FIG. 8A, first formulation 805 is a preparatory formulation and second formulation 810 is a therapeutic formulation, and the environment surrounding delivery device 800 is fluid (e.g., biological matter, digestive matter). In this example, a rate of degradation of plug 705 is designed to be faster than a rate of degradation of plug 710, such that plug 710 blocks fluid from passing into chamber 612 from the environment surrounding shell 605 longer than plug 705 blocks fluid from passing into chamber 611 from the environment surrounding shell 605. After placement in the body (e.g., at a target location), delivery device 800 is exposed to fluid. Plug 705 is designed to begin degrading after exposure to fluid at a target location (e.g., substantially immediately, or when a pH of fluid is within a predefined range or crosses a predefined threshold, or after a predefined time period, or after another defined condition occurs). Dependent on the rate of degradation of plug 705, fluid will eventually breach plug 705 and enter chamber 611. The preparatory formulation (first formulation 805) diffuses from chamber 611 to the environment through breached plug 705 to prepare the environment for delivery of the therapeutic formulation (second formulation 810).

The preparatory formulation can be, or can include, for example, a permeation enhancer, an enzyme blocker, a peptide that permeates through mucosa, an antiviral drug such as a protease inhibitor, a disintegrant or superdisintegrant, a pH modifier, a vasodilator, or other formulation.

Continuing with the first example with respect to FIG. 8A, when fluid enters chamber 611, plug 720 begins to degrade (e.g., substantially immediately, or when a pH of fluid is within a predefined range or crosses a predefined threshold, or after a predefined time period, or after another defined condition occurs). Dependent on the rate of degradation of plug 720, fluid will eventually breach plug 720 and enter chamber 612. Subsequently, the therapeutic formulation (second formulation 810) diffuses from chamber 612 to chamber 611 and then to the environment. Note that orifice 725 can be positioned at any location between chamber 611 and chamber 612. Thus, for example, orifice 725 (and thus plug 720) can be positioned at a portion of wall 615 furthest from plug 705 to delay degradation of plug 720 until a substantial percentage of first formulation 805 has diffused through breached plug 705 to the environment, such as in a case in which it is preferable for the preparatory formulation to have a maximum time of action in the environment before the therapeutic formulation is present in the environment. For another example, in a case in which it is preferable for the preparatory formulation to be present in the environment concurrently with the therapeutic formulation, orifice 725 (and thus plug 720) can be positioned nearer to plug 705.

In the first example with respect to delivery device 800 in FIG. 8A, the rate of degradation of plug 710 is designed to be slower than a time period during which (a) plug 705 degrades sufficiently to allow fluid to pass into chamber 611 and (b) plug 720 subsequently (after fluid passes into chamber 611) degrades sufficiently to allow fluid to pass into chamber 612. After plug 710 is breached, the therapeutic formulation (second formulation 810) diffuses by passing through chamber 611 and then through breached plug 705, and also by passing through breached plug 710. An example of an elution profile graph for the first example with respect to delivery device 800 is as follows (Graph 1).

In a second example with respect to delivery device 800 in FIG. 8A, plug 710 is non-degradable, or has a rate of degradation such that a time period to a breach of plug 710 due to exposure to fluid (from the environment and/or from chamber 612) is designed to be after the therapeutic formulation (second formulation 810) has substantially diffused from chamber 612 through chamber 611 to the environment. An example of an elution profile graph for the second example with respect to delivery device 800 is as follows (Graph 2).

In a third example with respect to delivery device 800 in FIG. 8A, plug 710 is designed to resist breach from degradation for a period of time longer than an expected time to breach of plug 705, such as minutes, hours, days, weeks, months, or longer, and plug 720 is non-degradable, or degrades at a rate slower than plug 710. In this example, first formulation 805 and second formulation 810 can include substantially the same constituent materials, or can include different materials. For example, second formulation 810 can be a subsequent dose of first formulation 805 (e.g., for an immunization booster, or for multiple dosing with a single delivery device), can include a different agent to treat different aspects of a condition treated by one or more agent(s) in first formulation 805, can be a different agent for a different purpose than agents in first formulation 805 or to treat different conditions than agents in first formulation 805, or can be a formulation including first formulation 805 along with other agents. An example of an elution profile graph for the third example with respect to delivery device 800 is as follows (Graph 3), where second formulation 810 diffusion is shown corresponding to different times for the breach of plug 710, either approximately at breach of plug 705 (ex. A), or after breach of plug 705 (ex. B, ex. C)).

FIG. 8B illustrates a delivery device 850 including the shell 605 configuration of FIG. 7D, with a third formulation 815 disposed in chamber 611 and a fourth formulation 820 disposed in chamber 612. Plug 715 can be designed to degrade after exposure to fluid (e.g., substantially immediately, or when a pH of fluid is within a predefined range or crosses a predefined threshold, or after a predefined time period, or after another trigger condition occurs). In such a case, dependent on the rate of degradation of plug 715, fluid will eventually breach plug 715 and enter chamber 611. Third formulation 815 diffuses from chamber 611 to the environment through breached plug 715.

In a first example with respect to FIG. 8B, plug 735 is non-degradable, and when fluid enters chamber 611 through breached plug 715, plug 710 begins to degrade (e.g., substantially immediately, or when a pH of fluid is within a predefined range or crosses a predefined threshold, or after a predefined time period, or after another trigger condition occurs). Dependent on the rate of degradation of plug 710, fluid will eventually breach plug 710 and enter chamber 612. Fourth formulation 820 diffuses from chamber 612 to cavity 610 and then to the environment through breached plug 715.

In a second example with respect to FIG. 8B, plug 735 is degradable, and when fluid enters chamber 611 through breached plug 715, plug 735 begins to degrade (e.g., substantially immediately, or when a pH of fluid is within a predefined range or crosses a predefined threshold, or after a predefined time period, or after another trigger condition occurs). Dependent on the rate of degradation of plug 735, fluid will eventually breach plug 735 and enter chamber 612. Fourth formulation 820 diffuses from chamber 612 to chamber 611 and then to the environment through breached plug 735, and possibly also through beached plug 710 as described with respect to the first example with respect to FIG. 8B. Note that orifice 730 (FIG. 7D) can be positioned at any location between chamber 611 and chamber 612. Thus, for example, orifice 730 (and thus plug 735) can be positioned at a portion of wall 615 furthest from plug 705 to delay degradation of plug 735 until a substantial percentage of third formulation 815 has diffused through breached plug 715 to the environment. For another example, orifice 730 (and thus plug 735) can be positioned nearer to plug 715.

In other embodiments of FIG. 8A, shell 605, or a portion thereof, can be degradable, alternatively or additionally to plugs 705, 710, 720. In other embodiments of FIG. 8B, shell 605, or a portion thereof, can be degradable, alternatively or additionally to plugs 710, 715, 735. Further, in any of the foregoing embodiments or other embodiments, wall 615, or a portion thereof, can be degradable. Accordingly, a desired elution profile can be determined, and then implemented, using a combination of degradable components (e.g., shell, wall(s), and/or plug(s)).

As can be seen from FIGS. 8A and 8B and the descriptions thereof, a delivery device such as described herein can be provided for vaccination, for multiple dosing, for delivering multiple agents at different times with a single device, for increasing dosages over time, for changing agents over time according to a therapeutic plan, and so forth.

A noted above, a wall (e.g., wall 615) defining multiple chambers within a cavity can itself be degradable. Thus, a degradable material can be used to construct a wall additionally or alternatively to using a plug in the wall. For example, for a wall that includes a plug, the plug can be designed to degrade (be breached) more quickly than the wall is designed to degrade (be breached), allowing an elution profile to be designed such that a small amount of formulation is diffused from a chamber as the plug begins to degrade, and then substantially completely degrades, followed by an increasing amount of diffusion of formulation from the chamber as the wall begins to degrade, then substantially completely degrades.

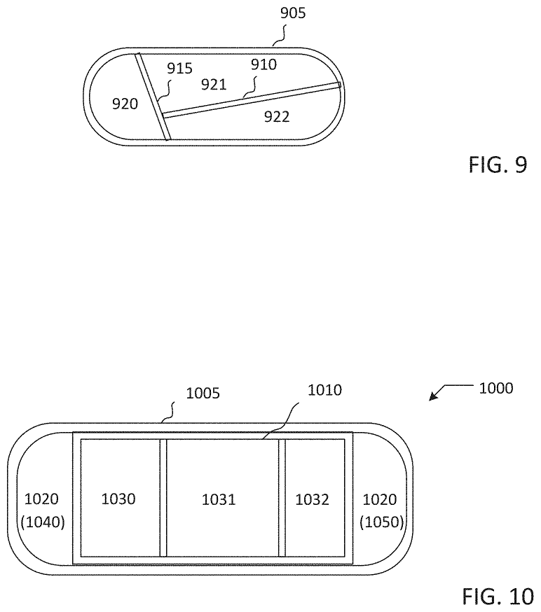

FIG. 9 illustrates an example of a shell 905 in which two walls 910, 915 divide a cavity defined by shell 905 into three chambers 920, 921, 922, illustrating that walls can be positioned at any point and in any orientation within a shell. Degradable plugs and/or degradable wall materials can be used to prevent, minimize, allow, or promote diffusion of formulations from one or more of chambers 920, 921, 922 in accordance with a desired elution profile, as discussed above.

FIG. 10 illustrates an example of a delivery device 1000 incorporating a shell 1005 and a wall-and-chamber structure 1010 manufactured independently of each other, such that structure 1010 can be placed into a cavity 1020 defined by shell 1005. Structure 1010 can be movable within cavity 1020, or can be fitted within cavity 1020 so as to not be generally movable therein. Degradable plugs and/or degradable wall materials can be used to prevent, minimize, allow, or promote diffusion of formulations from shell 1005 and from one or more chambers (e.g., chambers 1030, 1031, 1032) defined by structure 1010 in accordance with a desired elution profile, as discussed above. Further, positioning of structure 1010 within cavity 1020 can define additional chambers (e.g., chambers 1040, 1050) within cavity 1020 around structure 1010, depending on the size, shape, and placement of structure 1010 within cavity 1020.

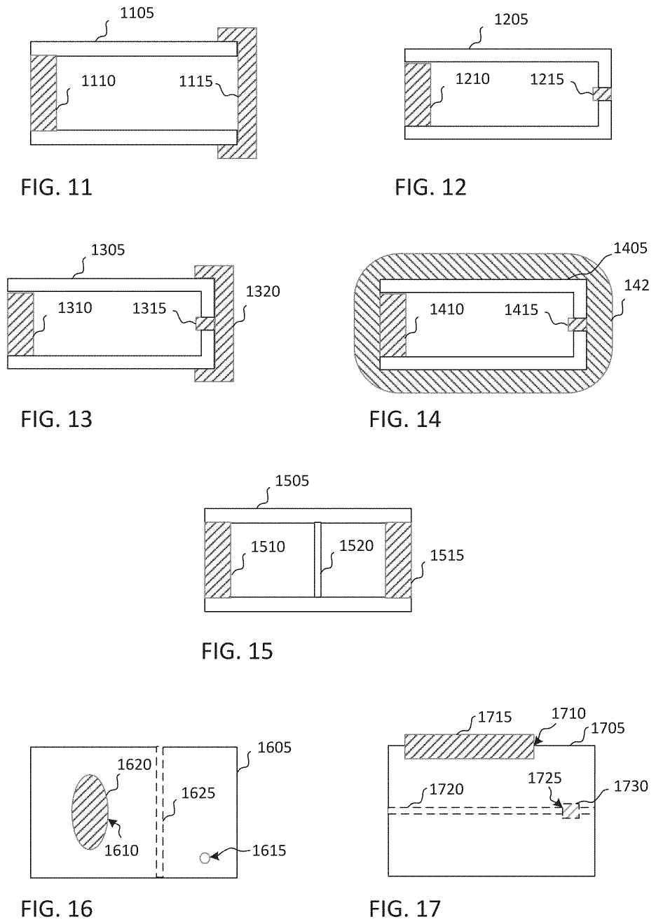

FIGS. 11-15 illustrate several examples of embodiments of delivery devices including multiple orifices, each shown in a slice view (e.g., in an x-y plane of the x-y-z domain along the lengthwise axis (x-axis) of the shell of the delivery device).

FIG. 11 illustrates a shell 1105 which is fully open at both ends, with a plug 1110 fitted into one end of shell 1105 and a plug 1115 fitted over another end of shell 1105. FIG. 12 illustrates a shell 1205 which is fully open at one end with a plug 1210 fitted into the fully open end. Shell 1205 is partially open at another end and has a plug 1215 fitted into the partially open end. FIG. 13 illustrates a shell 1305 which is fully open at one end with a plug 1310 fitted into the fully open end, is partially open at another end with a plug 1315 fitted into the partially open end, and with a plug 1320 disposed over plug 1315 and over the partially open end of shell 1305.

FIG. 14 illustrates a shell 1405 which is fully open at one end with a plug 1410 fitted into the fully open end, and is partially open at another end with a plug 1415 fitted into the partially open end. A coating 1420 covers the entirety of the exteriors of shell 1405 and plugs 1410, 1415. Coatings are discussed in detail below.

FIG. 15 illustrates a shell 1505 which is fully open at two ends with two plugs 1510, 1515, fitted one into each end. Shell 1505 includes a wall 1520 in a cavity defined by shell 1505.

FIG. 16 illustrates a shell 1605 viewed from outside shell 1605 (i.e., not in cross-section). Shell 1605 defines two orifices 1610, 1615 viewable from a same perspective (e.g., along a same face, along a same plane, or in different planes or surfaces but both viewable from a same point exterior to shell 1605). A plug 1620 is disposed in orifice 1610, and orifice 1615 is left open. A wall 1625 is disposed internal to shell 1605.

FIG. 17 illustrates a shell 1705 viewed from outside shell 1705. Shell 1705 defines an orifice 1710 in which a plug 1715 is disposed. A wall 1720 disposed internal to shell 1705 defines an orifice 1725 in which a plug 1730 is disposed.

It can be understood from the examples illustrated and described above with respect to FIGS. 1A-1E, 2A-2D, 3A-3J, 4A-4D, 5A-5D, 6A-6C, 7A-7D, 8A, 8B, 9-17 and subsequent discussions that a delivery device can be designed in accordance with embodiments of the present disclosure to deliver a formulation (or agent thereof) in accordance with a desired elution profile, and can further be designed for delivery at a particular target site. These figures illustrate a few of the many combinations of shells, walls, and plugs encompassed by the present disclosure. Note that a delivery device can have any cross-sectional shape as viewed from any direction, and the cross-sectional shape can vary along a length of the delivery device. FIGS. 3A-3J provide a few examples of shapes as viewed from an end of a delivery device; any other shape is also within the scope of the present disclosure.

As introduced with respect to FIG. 1E, a delivery device can be equipped with a pointed portion, such as to penetrate through a material at a target delivery site.

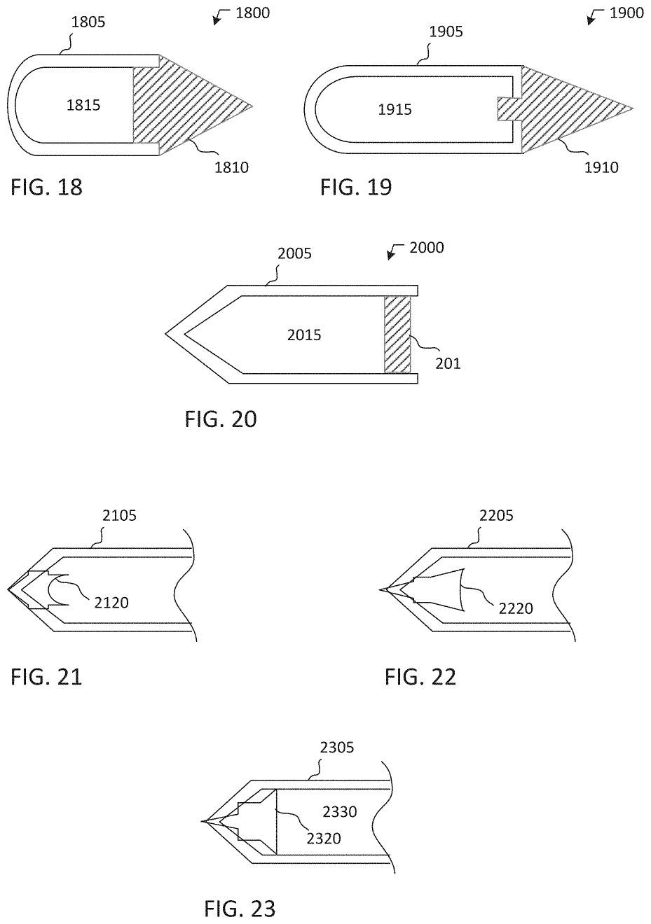

FIGS. 18-23 provide illustrations of additional examples in which a delivery device includes a point, each shown in a slice view (e.g., in an x-y plane of the x-y-z domain along the lengthwise axis (x-axis) of the shell of the delivery device).

The plugs in FIGS. 18-20 each are constructed of materials such that the plug can substantially retain its shape at least during a first portion of a traversal of a corresponding delivery device through matter at a target delivery site. For example, a delivery device can be delivered through tissue in a body, such as through a membrane, into or through a wall of an organ (e.g., heart, intestine, stomach, brain, reproductive organ, or other organ), into or through a muscle, or into or through connective tissue, and the plug is constructed in a manner and with materials to promote motion into and/or through the tissue.

FIG. 18 illustrates a delivery device 1800 including a shell 1805 having a fully open end in which a plug 1810 is disposed.

FIG. 19 illustrates a delivery device 1900 including a shell 1905 having a fully open end in which a plug 1910 is disposed.

FIG. 20 illustrates a delivery device 2000 including a shell 2005 having a fully open end in which a plug 2010 is disposed. In this example, shell 2005 has a pointed end, for example as described with respect to FIG. 1E. The pointed end of shell 2005 is constructed of materials such that the pointed end can substantially retain its shape at least during a first portion of a traversal of delivery device 2000 through matter at a target delivery site. For example, delivery device 2000 can be delivered through tissue in a body, such as through a membrane, into or through a wall of an organ (e.g., heart, intestine, stomach, brain, reproductive organ, or other organ), into or through a muscle, or into or through connective tissue, and shell 2005 is constructed in a manner and with materials to promote motion into and/or through the tissue. The pointed end of shell 2005 can be constructed of different materials than other portions of shell 2005, such as to increase a resistance of the pointed end to breakage, or shell 2005 can be constructed of a same material or combination of materials throughout the entirety of shell 2005 including the pointed end. In one or more embodiments, a resistance to breakage at the pointed end of shell 2005 is increased by increasing a thickness of material at the pointed end. In one or more embodiments, a resistance to breakage at the pointed end of shell 2005 is increased by adding a coating over the pointed end, such as by adding a metal or carbon film coating over the pointed end.

FIGS. 21-23 illustrate increasing a resistance to breakage at the pointed end of a shell (e.g., shell 2005) by adding a tip constructed of a hard material, such as a metal, carbon, a composite, or other hard material. Such a tip can be a thin flat piece, or can have a generally consistent profile around an axis, or can have a varied profile around an axis.

FIG. 21 illustrates a shell 2105 in which a tip 2120 is embedded within a material at an end of shell 2105, such as to reinforce the material at the end of shell 2105.

FIG. 22 illustrates a shell 2205 in which a tip 2220 is embedded within and protrudes through a material at an end of shell 2205.

FIG. 23 illustrates a shell 2305 in which a tip 2320 is embedded within and protrudes through a material at an end of shell 2305. Tip 2320 extends fully across a cavity 2330 formed by shell 2305 and touches at least a portion of shell 2305 within cavity 2330, such as to stabilize tip 2320 in an orientation and/or at a position within cavity 2330.

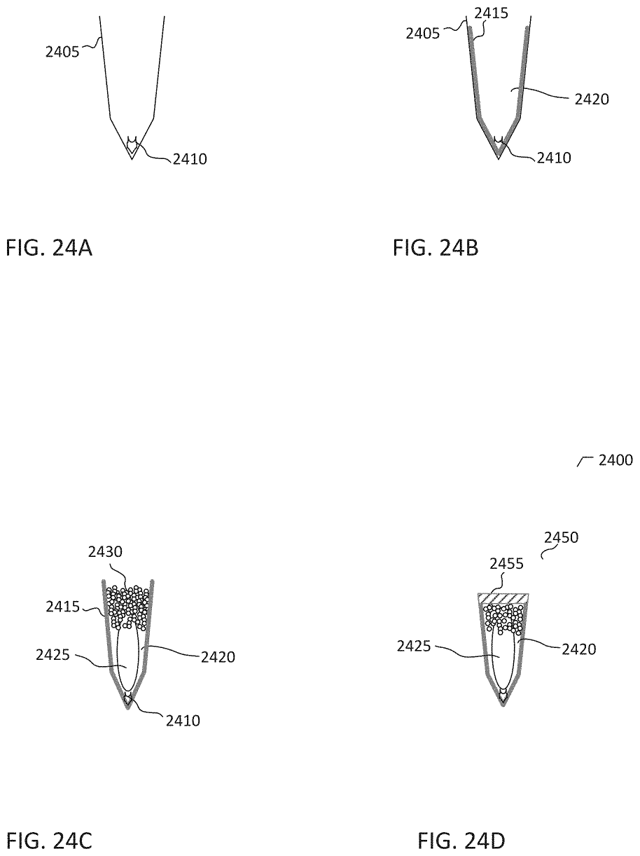

FIGS. 24A-24C illustrate an example of a technique for forming a delivery device 2400 (e.g., an embodiment of shell 2105 of FIG. 21). FIG. 24A illustrates a shell mold 2405 (e.g., an injection mold) in which a tip 2410 is positioned. FIG. 24B illustrates a shell frame 2415 molded in shell mold 2405, and tip 2410 embedded in shell frame 2415. Shell frame 2415 defines a cavity 2420. FIG. 24C illustrates a pill-shaped formulation 2425 disposed in cavity 2420, and an optional layer of insulating material 2430 disposed over formulation 2425. Examples of insulating material 2430 include sucrose, maltose, PEO, and polyvinyl alcohol (PVA).

FIG. 24D illustrates delivery device 2400 after shell frame 2415 is sealed (e.g., heat staked or otherwise heat sealed). The resulting seal closes cavity 2420, leaving a plug 2455 formed across an end of a shell 2450 of delivery device 2400.

A delivery device can itself be a payload for another delivery device. For example, delivery device 2400 can be contained inside a capsule, or inside a spring-loaded or other mechanical mechanism within a housing which mechanism ejects delivery device 2400 out of the housing (e.g., into human tissue).

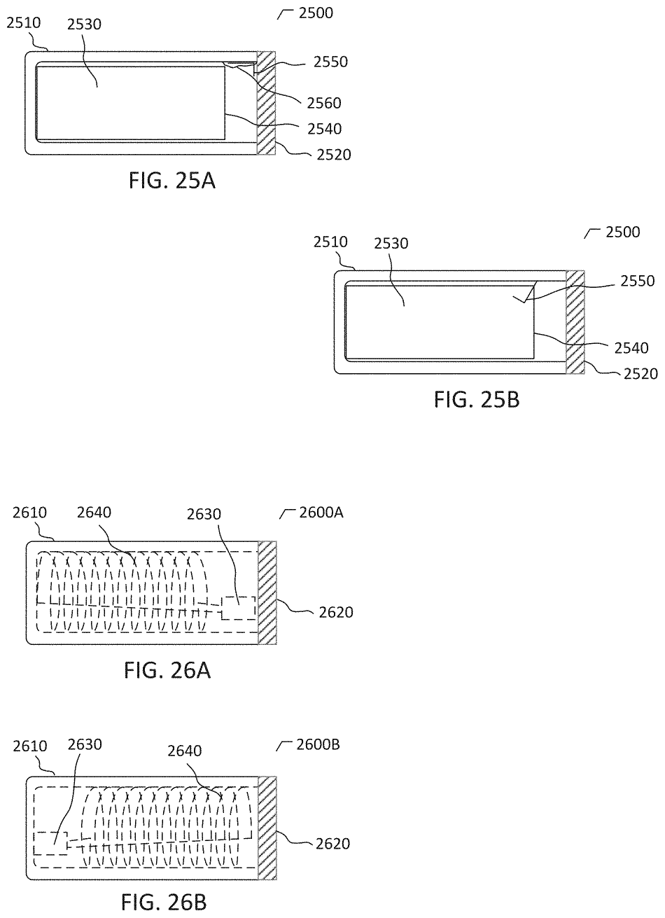

FIGS. 25A, 25B illustrate a payload which itself is a delivery device in the form of a self-contained container.

FIG. 25A illustrates a delivery device 2500 including a shell 2510 with a plug 2520 closing an end of shell 2510. A payload 2530 is disposed within shell 2510, and is protected from an environment outside of shell 2510 until plug 2520 degrades sufficiently to allow a breach of plug 2520. Payload 2530 is a self-contained container, which can include a fluid. A seal cap 2540 is disposed over payload 2530 and is maintained in a taut configuration by payload 2530. For example, seal cap 2540 can be aluminum foil or a polymer material which is adhered by an adhesive or by heat stake or by vibration stake to a body portion of payload 2530. Seal cap 2540 inhibits, minimizes, or prevents fluid from entering payload 2530.

Delivery device 2500 further includes a puncture mechanism, such as the puncture mechanism 2550 depicted in FIGS. 25A, 25B. Puncture mechanism 2550 is held in a pre-release form by a degradable overmold 2560. For example, overmold 2560 can be formed from a sugar composition, a PEO composition, or another substance or composition which degrades in the presence of fluid generally, or in the presence of a specific chemical composition.

When plug 2520 is breached, fluid passes through plug 2520 and reaches an interior of shell 2510. Seal cap 2540 is resistant to the fluid. Overmold 2560 degrades in the presence of the fluid, and eventually is sufficiently degraded to allow puncture mechanism 2550 to release. As illustrated in FIG. 25B, puncture mechanism 2550, once released, punctures seal cap 2540 of payload 2530, allowing fluid to enter (or exit) payload 2530. Payload 2530 optionally can also have a designed degradation profile. Examples of designed degradation profiles are found throughout the present disclosure. For example, payload 2530 can in some embodiments itself be a delivery device such as an embodiment of a delivery device of the present disclosure.

Overmold 2560 can be designed to withstand degradation for a period of time. For example, a thickness or composition of overmold 2560 can be adjusted to provide a design time period of degradation in seconds or minutes or hours.

FIGS. 26A, 26B illustrate examples of embodiments of delivery devices containing electronics. According to various embodiments, the components included or associated with electronics in an embodiment of the present disclosure can correspond to one or more of a receiver, a transmitter, a processor, a digital signal processor, power management circuitry, a battery, and/or a battery charger interface for charging remotely, or other circuitry. It is to be understood that processors and various circuitry may include instructions, such as hard-wired instructions, firmware, or software, for controlling the processor or circuitry in a desired fashion.

FIG. 26A illustrates a delivery device 2600A including a shell 2610 with a plug 2620 closing an end of shell 2610. Delivery device 2600A can optionally include a payload (e.g., a therapeutic formulation, payload 2530 in FIGS. 26A, 26B with a corresponding puncture mechanism incorporated with delivery device 2600A, or other payload). Plug 2620 degrades sufficiently in a target environment to allow a breach of plug 2620, and fluid can then enter delivery device 2600A. Delivery device 2600A includes electronics 2630 and an antenna 2640 electrically coupled to electronics 2630. Electronics 2630 detects the presence of fluid, such as by detecting a change in resistance or capacitance between two electrodes, and can perform a task such as transmitting a signal to an external device by way of antenna 2640. In an embodiment, delivery device 2600A can be used to detect a presence of a chemical composition that degrades plug 2620, such as to detect a tumor site, to detect an area which is bleeding, or to indicate the arrival of delivery device 2600A at a location having a pH level at, above, or below which plug 2620 is designed to degrade.

FIG. 26B is similar to FIG. 26A except that a delivery device 2600B includes electronics 2630 positioned at an end of delivery device 2600B away from plug 2620, in comparison to electronics 2630 being positioned near plug 2620 as illustrated in FIG. 26B. In FIG. 26B, a formulation (not shown) can be disposed within delivery device 2600B; after plug 2620 is breached, the formulation can begin to degrade upon exposure to fluid entering delivery device 2600B through breached plug 2620. Eventually, electronics 2630 will be exposed to fluid by degradation of the formulation, electronics 2630 can detect the fluid, and can transmit a signal through antenna 2640 indicating that fluid is detected. Such a configuration can be used, for example, to signal that the formulation has mostly diffused out of delivery device 2600B. In an embodiment similar to the embodiments illustrated in FIGS. 26A, 26B, electronics 2630 can be positioned at a point between the ends of delivery device 2600A or 2600B, respectively, such as to provide a signal when a portion of the formulation has diffused out (e.g., when approximately 30% has diffused out, or when approximately 50% has diffused out, or when approximately 75% has diffused out) to provide information external to the subject that the drug is being delivered, or that it is time for a new dose.

A sensor can be used to detect fluid and trigger a circuit in electronics included in a delivery device, to wake up (e.g., transition from a low-power state to a higher-power state) the electronics to perform a function, such as log an environmental condition, initiate delivery of a formulation, or transmit a signal.

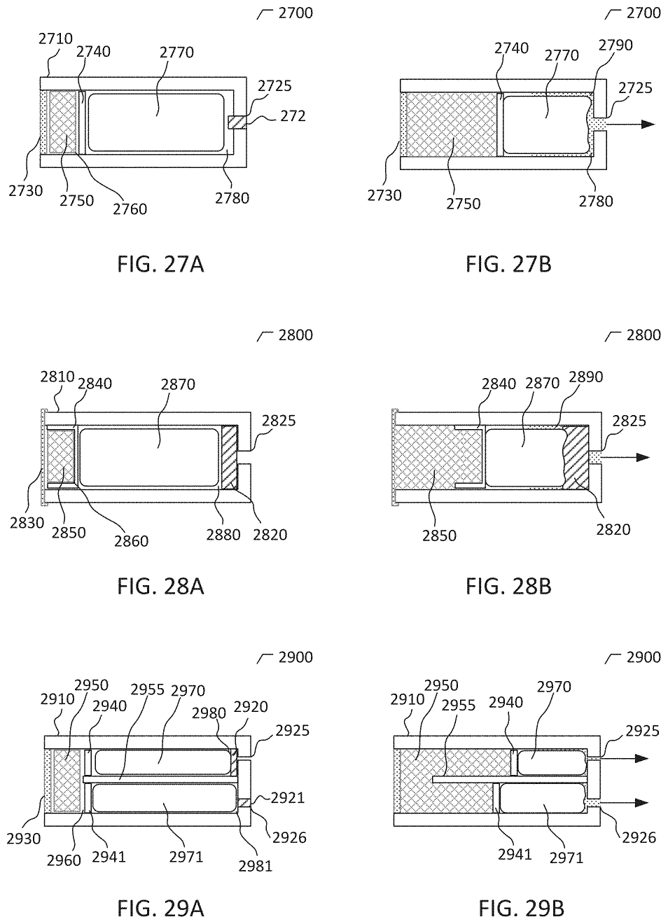

The delivery devices described above allow for passively diffusing the contents of the delivery device to the environment. In some cases, it is desirable to increase a rate of diffusion, such as through forcing the contents out under pressure. For example, the delivery device can incorporate a technique in which a pressure internal to the delivery device is greater than a pressure external to the delivery device, so that the contents of the delivery device are pushed or squeezed out of the delivery device under pressure. FIGS. 27A, 27B, 28A, 28B, 29A, 29B, 30A, 30B, 31A, 31B, 32A, 32B illustrate examples of delivery devices including pumps.

In FIGS. 27A, 27B, a delivery device 2700 includes a shell 2710, a plug 2720 in an orifice 2725 defined by shell 2710, a plug 2730, a piston 2740, and an expander 2750. In this embodiment, plug 2720 is designed to degrade upon exposure to defined environmental conditions, such as exposure to fluid or exposure to a particular chemical composition or family of chemical compositions, or exposure to an environment having a pH value within a range or above or below a threshold. Plug 2730 is designed to withstand degradation under the defined environmental conditions in which plug 2720 degrades. Plug 2730 is designed to allow passage of fluid through plug 2730. In one or more embodiments, plug 2730 defines multiple orifices. In one or more embodiments, plug 2730 is, or includes, a mesh or a membrane.

Piston 2740 (and pistons in other embodiments of the present disclosure) can be degradable or non-degradable; however, generally, a piston in accordance with the present disclosure is structured to withstand degradation for a time sufficient to allow a formulation disposed within the delivery device to fully diffuse out of the delivery device in accordance with a desired elution profile. Examples of degradable materials that can be used in a piston include PLA, PLGA, Mg, or a combination of two or more of the foregoing.

Expander 2750 is, or includes, a hydrogel and a salt. An example of a hydrogel is PEO and/or polyacrylamide loaded with salts. In its initial state within delivery device 2700, expander 2750 is dry (e.g., dehydrated) and sits within a cavity 2760 defined by shell 2710, piston 2740, and plug 2730, exerting minimal or no force against piston 2740. When delivery device 2700 is exposed to fluid, fluid enters delivery device 2700 by way of plug 2730 and reaches expander 2750, which begins to expand and thus exert force against piston 2740. Meanwhile, plug 2720 begins to degrade and eventually breaches, allowing fluid to enter delivery device 2700 through breached plug 2720.

A formulation 2770 is disposed in a cavity 2780 defined by shell 2710 and piston 2740. As fluid enters delivery device 2700 through breached plug 2720, fluid enters cavity 2780, and begins to degrade formulation 2770. A fluidized formulation 2790 (FIG. 27B) is formed in cavity 2780 as formulation 2770 degrades. Fluidized formulation 2790 can have various consistencies depending on a constitution of formulation 2770. For example, fluidized formulation 2790 can have high fluid content or low fluid content. For another example, fluidized formulation 2790 can include large particles, small particles, or a variety of particle sizes. For a further example, fluidized formulation 2790 can be hydrophobic or hydrophilic.

FIG. 27B illustrates that fluidized formulation 2790 diffuses through orifice 2725, as indicated by the arrow. Although illustrated as diffusion through orifice 2725 with plug 2720 fully degraded, diffusion can begin before complete degradation of plug 2720, after plug 2720 is breached.

In some embodiments, delivery device 2700 is designed for plug 2720 to be breached at a target location before expander 2750 begins to expand, such that fluidized formulation 2790 initially diffuses from cavity 2780 passively until expander 2750 expands and exerts force on piston 2740, causing piston 2740 to exert force on formulation 2770 and thus force fluidized formulation 2790 out through orifice 2725 (see, e.g., elution profile Graph 4 illustrating diffusion with respect to formulation 2770). In other embodiments, delivery device 2700 is designed for plug 2720 to be breached at a target location after expander 2750 begins to expand, such that by the time plug 2720 is breached, piston 2740 exerts pressure on formulation 2770 and thus forces fluidized formulation 2790 out through orifice 2725 (see, e.g., elution profile Graph 5 illustrating diffusion with respect to formulation 2770). In either case, at some point, fluidized formulation 2790 is forced out of shell 2710 through orifice 2725 under pressure due to the force of expander 2750 against piston 2740 and the resulting force of piston 2740 against formulation 2770.

In one or more embodiments, delivery device 2700 diffuses fluidized formulation 2790 at a semi-constant rate after full degradation of plug 2720 and expansion of expander 2750 begins, until piston 2740 can move no further.

The rate at which a fluidized formulation is diffused can be designed by selection of materials of an expander. For example, a salt content of expander 2750 can be increased to increase force against piston 2740.

In FIGS. 28A, 28B, a delivery device 2800 includes a shell 2810, a plug 2820, an orifice 2825 defined by shell 2810, a plug 2830, a piston 2840, an expander 2850, and a formulation 2870 disposed in a cavity 2880 defined by shell 2810, plug 2820, and piston 2840. In this embodiment, plug 2820 is a dry hydrogel when initially disposed within delivery device 2800. After delivery device 2800 is exposed to fluid, fluid enters through orifice 2825 and is gradually absorbed by plug 2820, which expands due to the absorption. Fluid in the hydrogel degrades formulation 2870 across an interface between plug 2820 and formulation 2870, forming a fluidized formulation 2890 (FIG. 28B) which is partially contained within the hydrogel of plug 2820. Until piston 2840 begins to move, fluidized formulation 2890, to the extent that it is not contained within plug 2820 or blocked by plug 2820, passively diffuses out of delivery device 2800 through orifice 2825.

Plug 2830 is designed to withstand degradation under environmental conditions at a target environment, and is further designed to allow passage of fluid. In one or more embodiments, plug 2830 defines multiple orifices to allow passage of fluid. In one or more embodiments, plug 2830 is, or includes, a mesh or a membrane to allow passage of fluid.

Expander 2850 is, or includes, a hydrogel and a salt. In its initial state within delivery device 2800, expander 2850 is dry and sits within a cavity 2860 defined by piston 2840 and plug 2830, exerting minimal or no force against piston 2840. When delivery device 2800 is exposed to fluid, fluid enters delivery device 2800 by way of plug 2830 and reaches expander 2850, which begins to expand and thus exert force against piston 2840. As piston 2840 moves due to the force exerted against it, piston 2840 in turn exerts a force against formulation 2870, and formulation 2870 in turn exerts force against plug 2820. The force against plug 2820 squeezes fluid (e.g., fluid from the environment and fluidized formulation 2890) out of the hydrogel in plug 2820, and fluid is diffused through orifice 2825 under pressure. The rate at which a fluidized formulation is diffused can be designed by selection of materials of plug 2820 and materials of expander 2850. For example, a salt content of expander 2850 can be increased to increase force against piston 2840 and overcome a resistance of the hydrogel of plug 2820.

In FIGS. 29A, 29B, a delivery device 2900 includes a shell 2910, orifices 2925 and 2926 defined by shell 2910, a plug 2920 disposed at orifice 2925, a plug 2921 disposed at orifice 2926, a plug 2930, two pistons 2940, 2941, an expander 2950, and a wall 2955. Delivery device 2900 contains two formulations, a formulation 2970 disposed in a cavity 2980 formed by shell 2910, piston 2940, wall 2955, and plug 2920, and a formulation 2971 disposed in a cavity 2981 formed by shell 2910, piston 2941, and wall 2955.

Plug 2920 can include materials similar to materials of plug 2921, or materials different from materials of plug 2921. Either or both of plugs 2920, 2921 can be degradable (e.g., similar in materials and/or function to plug 2720 of FIG. 27A). Either or both of plugs 2920, 2921 can be, or can include, a hydrogel (e.g., similar in materials and/or function to plug 2820 of FIG. 28A). Either or both of plugs 2920, 2921 can include one or more degradable materials and a hydrogel, or a hydrogel layer.

Plug 2930 is designed to withstand degradation under environmental conditions at a target environment, and is further designed to allow passage of fluid. In one or more embodiments, plug 2930 defines multiple orifices to allow passage of fluid. In one or more embodiments, plug 2930 is, or includes, a mesh or a membrane to allow passage of fluid.

Expander 2950 is, or includes, a hydrogel and a salt. In its initial state within delivery device 2900, expander 2950 sits within a cavity 2960 defined by shell 2910, pistons 2940, 2941, and plug 2930, exerting minimal or no force against pistons 2940, 2941. When delivery device 2900 is exposed to fluid, fluid enters delivery device 2900 by way of plug 2930 and reaches expander 2950, which begins to expand and thus exert force against pistons 2940, 2941.

As piston 2940 moves due to the force exerted against it by expander 2950, piston 2940 in turn exerts a force against formulation 2970, and formulation 2970 in turn exerts force against plug 2920, and a fluidized formulation resulting from elution of fluid and formulation 2970 is diffused through orifice 2925 under pressure. Meanwhile, as piston 2941 moves due to the force exerted against it by expander 2950, piston 2941 in turn exerts a force against formulation 2971, and a fluidized formulation resulting from elution of fluid and formulation 2971 is diffused through orifice 2926 under pressure.

Expander 2950 can expand in a manner such that force is applied somewhat evenly against pistons 2940, 2941 as compared to each other even if, as illustrated in FIG. 29B, formulations 2970, 2971 degrade at a different rate.

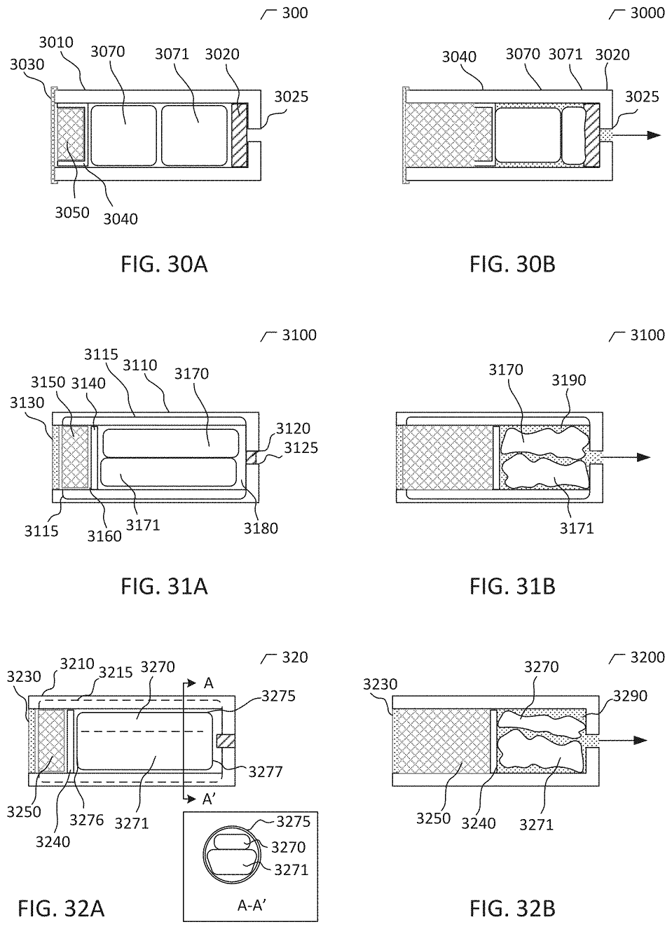

In FIGS. 30A, 30B, a delivery device 3000 is constructed in a manner similar to delivery device 2800 in FIG. 28A, except that two formulations 3070, 3071 are disposed in a cavity defined by a shell 3010, a piston 3040, and a plug 3020. In this embodiment, plug 3020 is a hydrogel. In other embodiments, plug 3020 can be similar to any of the other plugs described herein. As described above with respect to expander 2850 in FIG. 28A, an expander 3050 expands when exposed to fluid and exerts force against piston 3040 which in turn exerts force against formulation 3070. The force against formulation 3070 results in force by formulation 3070 against formulation 3071, causing a fluidized formulation formed by elution of formulation 3071 (and formulation 3070) with fluid from the environment to be diffused from delivery device 3000 under pressure. The use of two formulations 3070, 3071 arranged as illustrated allows, for example, for increased dosing using a same formulated tablet size and constituency, or for staggered dosing of different formulations.

In FIGS. 31A, 31B, a delivery device 3100 is constructed in a manner similar to delivery device 2700 in FIG. 27A, except that a shell 3110 incorporates one or more channels 3115 which extend from a cavity 3160 lengthwise along delivery device 3100 towards an end of shell 3110 that includes a plug 3120 disposed in an orifice 3125. Delivery device 3100 further includes two formulations 3170, 3171 disposed alongside each other in a cavity 3180 defined by shell 3110 and a piston 3140. Shell 3110, piston 3140, and a plug 3130 define a cavity 3160.

In the illustration of FIG. 31A, channel(s) 3115 extend along nearly a full length of cavity 3160 and nearly a full length of cavity 3180. In one or more embodiments, one or more of channel(s) 3115 do not extend as far along cavity 3160 and/or cavity 3180 as illustrated. An expander 3150 (e.g., a hydrogel plus a salt) disposed in cavity 3160 expands upon exposure to fluid entering through plug 3130 and/or orifice 3125 and exerts force against piston 3140. Fluid entering by way of plug 3130 is absorbed in part by expander 3150, and is also transmitted (e.g., by wicking, or by pushing due to expansion of expander 3150) through channel(s) 3115 and into cavity 3180. Elution occurs between fluid from channel(s) 3115 and formulations 3170, 3171, as well as between formulations 3170, 3171 and fluid entering cavity 3180 through orifice 3125. Thus, as illustrated in FIG. 31B, formulations 3170, 3171 can form a fluidized formulation 3190 with fluid from channel(s) 3115 and fluids entering through orifice 3125, where the fluidized formulation can be in contact along more of a length of formulations 3170, 3171 than would have been the case without channel(s) 3115, and therefore an elution process between formulations 3170, 3171 and the fluid(s) can occur relatively more rapidly.

In FIGS. 32A, 32B, a delivery device 3200 includes a shell 3210 optionally defining one or more channel(s) 3215. Delivery device 3200 includes a container 3275 containing formulations 3270, 3271. As illustrated in the cross-sectional view of container 3275 along lines A-A′ in FIG. 32A, formulations 3270, 3271 are circumferentially enclosed by container 3275. Container 3275 can be closed or sealed at one or both ends (e.g., one or both of end 3276 and end 3277), and/or one or both ends can be left partially open, and/or one or both ends can be fully open. In an embodiment, container 3275 is degradable and closed at both ends, such that container 3275 first degrades when exposed to fluid, and then formulations 3270, 3271 degrade after fluid breaches container 3275. FIG. 32B illustrates an example of formulations 3270, 3271 having partially degraded and container 3275 substantially degraded, forming a fluidized formulation 3290 around formulations 3270, 3271. Fluidized formulation 3290 diffuses out of delivery device 3200 under pressure exerted by a piston 3240 due to expansion of an expander 3250 after coming in contact with fluid crossing a plug 3230.

As discussed above (e.g., with respect to FIGS. 27A, 27B, 28A, 28B, 29A, 29B, 30A, 30B, 31A, 31B, 32A, 32B), a plug (e.g., respectively plug 2730, 2830, 2930, 3030, 3130, 3230) can include multiple holes designed to allow fluid to pass through the plug. Some examples are drilled holes, naturally occurring holes, holes formed by a mesh structure, and holes defined by a membrane.

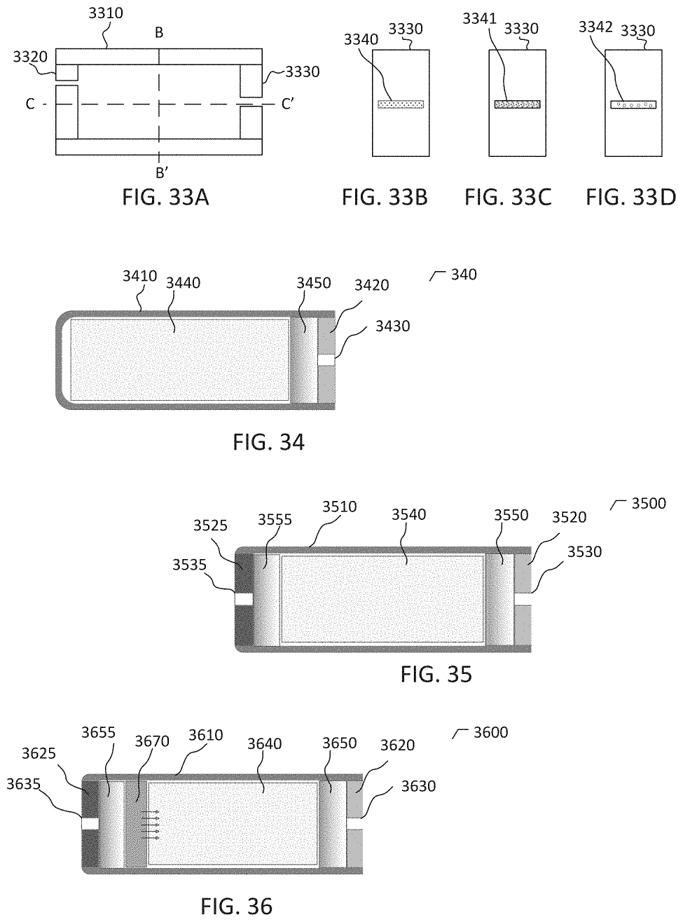

FIG. 33A illustrates a shell 3310 including an end portion 3320 and an end portion 3330. Portions of shell 3310 can be integrally formed. For example, end portion 3320 and/or end portion 3330 can be integrally formed with another portion of shell 3310, or can be separately formed and then affixed to another portion of shell 3310. For another example, shell 3310 can be formed in two portions which are then affixed to each other (e.g., two portions affixed to each other along line B-B′ or along line C-C′).

One or both of ends 3320, 3330 can include a plug having holes designed to allow fluid to pass through the plug. FIGS. 33B, 33C, 33D illustrate examples of such plugs, shown in end 3330. End 3320 can be structured similarly or differently. FIG. 33B illustrates a pattern of small holes through a plug 3340; FIG. 33C illustrates a mesh of holes through a plug 3341; and FIG. 33D illustrates a random position of holes through a plug 3342. Plugs 3340, 3341, 3342 illustrate a few examples of the variety of hole sizes and patterns. Additionally, although plugs 3340, 3341, 3342 are illustrated as being disposed in a small area of end 3330, in other embodiments, a plug can extend across a larger area of an end of a delivery device, and even fully across an end of a delivery device (e.g., such as illustrated with respect to FIGS. 27A, 27B, 28A, 28B, 29A, 29B, 30A, 30B, 31A, 31B, 32A, 32B). Therefore, a delivery device can be designed for slow or fast absorption of fluid into a hydrogel, or slow or fast elution with a formulation.

As described above, a hydrogel can be used in the presence of fluid by osmotic action to absorb fluid and thereby expand to exert pressure against a piston (e.g., fluid passing through a plug, such as plugs illustrated in FIGS. 27A, 27B, 28A, 28B, 29A, 29B, 30A, 30B, 31A, 31B, 32A, 32B, 33A, 33B, 33C, 33D or other plugs). Accordingly, embodiments of the present disclosure can be adapted to form an osmotic pump by the addition of a hydrogel expander. FIGS. 27A, 27B, 28A, 28B, 29A, 29B, 30A, 30B, 31A, 31B, 32A, 32B provide some examples of such embodiments; many other embodiments are within the scope of the present disclosure as will be apparent from the figures and descriptions herein.

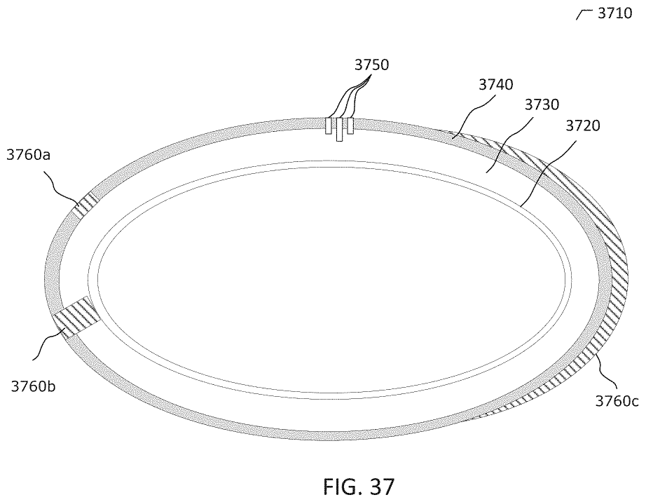

FIGS. 34, 35, 36 illustrate example designs of embodiments of delivery devices in accordance with the present disclosure. FIGS. 34, 35 illustrate examples of passive diffusion devices, whereas FIG. 36 illustrates an example of an osmotic pump delivery device.

In FIG. 34, a delivery device 3400 includes a shell 3410, and a plug 3420 defining an orifice 3430 or multiple orifices 3430 (or multiple orifices within the space illustrated as orifice 3430). A formulation 3440 is disposed within shell 3410. A plug 3450 is disposed within shell 3410 between formulation 3440 and plug 3420. Plug 3450 is, or includes, a molded dry (e.g., dehydrated) hydrogel when initially disposed in shell 3410. The hydrogel can include, for example, one of or a combination of PEO or polyacrylamide. A diffusion rate of formulation 3440 out of delivery device 3400 over time is related to a rate of elution of formulation 3440 with fluid entering delivery device 3400, together with a capacity of hydrogel plug 3450, and a total cross-sectional area of orifice(s) 3430 in plug 3420. For example, after the hydrogel plug 3450 is hydrated, the diffusion rate of formulation 3440 out of delivery device 3400 can be similar to (e.g., within 5%, within 10%, or within 20%) the rate of elution of formulation 3440 with fluid entering delivery device 3400, and both the diffusion rate and the rate of elution can be approximately proportional to the total cross-sectional area of orifice(s) 3430 in plug 3420.

In a first example of an embodiment of FIG. 34, shell 3410 is non-degradable. Shell 3410 can be constructed, for example, of a metal (e.g., stainless steel, titanium), a plastic, a polymer, or a combination thereof. In this first example of an embodiment of FIG. 34, plug 3420 is also non-degradable. Plug 3420 can be constructed, for example, of a metal (e.g., stainless steel, titanium), a plastic, a polymer, or a combination thereof. In this first example of an embodiment of FIG. 34, a total cross-sectional area of orifice 3430 (whether a single orifice or multiple orifices, or an effective cross-sectional area of passage through a membrane or mesh) defines a rate at which formulation 3440 diffuses out of delivery device 3400. Formulation 3440 will diffuse out of delivery device 3400 relatively quickly in an embodiment in which the cross-sectional area of orifice 3430 is large in comparison to an embodiment in which the cross-sectional area of orifice 3430 is much smaller (see, e.g., elution profile Graph 6, comparing elution profiles of different relative cross-sectional areas of orifice 3430, with larger to smaller cross-section areas shown in the progression A-B-C where A representatives the largest of the three). An area under the curve (AUC) will be substantially the same for each cross-sectional area of orifice 3430, but total diffusion time increases with a decrease in cross-sectional area of orifice 3430, and time-to-peak decreases with an increase in cross-sectional area of orifice 3430. The adjustment of orifice sizes to design a desired elution profile is also applicable to any other embodiment of the present disclosure.

In a second example of an embodiment of FIG. 34, shell 3410 is degradable. For example, shell 3410 is constructed of Mg, PLA, or PLGA, or a combination of two or more of the foregoing. In one or more embodiments, a designed time for degradation of shell 3410 in a target environment is greater than a designed time for formulation 3440 to diffuse out of delivery device 3400 by way of orifice(s) 3430. In one or more other embodiments, a designed time for degradation of shell 3410 in the target environment is approximately equal to or greater than the designed time for formulation 3440 to diffuse out of delivery device 3400. In the second example of FIG. 34, plug 3420 is also degradable. For example, plug 3420 is constructed of Mg, PLA, or PLGA, or a combination of two or more of the foregoing.

In a third example of an embodiment of FIG. 34, shell 3410 is degradable and plug 3420 is non-degradable.

In a fourth example of an embodiment of FIG. 34, shell 3410 is non-degradable and plug 3420 is degradable.

In a fifth example of an embodiment of FIG. 34, delivery device 3400 is similar to that described for the second example of an embodiment of FIG. 34, except that a membrane is disposed in orifice 3430.

In FIG. 35, a delivery device 3500 includes a shell 3510, a plug 3520 defining an orifice 3530 (or multiple orifices within the space illustrated as orifice 3530), a plug 3525 defining an orifice 3535 (or multiple orifices within the space illustrated as orifice 3535), a formulation 3540, a hydrogel plug 3550 disposed between formulation 3540 and plug 3520, and a hydrogel plug 3555 disposed between formulation 3540 and plug 3525. Delivery device 3500 operates in a fashion similar to delivery device 3400 of FIG. 34, except that elution and diffusion occur concurrently on both ends of delivery device 3500 when fluid enters shell 3510 by way of orifices 3530, 3535.

In one or more embodiments, a membrane is disposed in orifice 3530 or orifice 3535 of FIG. 35.

In FIG. 36, a delivery device 3600 includes a shell 3610, a plug 3620 defining an orifice 3630 (or multiple orifices within the space illustrated as orifice 3630), a plug 3625 defining an orifice 3635 (or multiple orifices within the space illustrated as orifice 3635), a formulation 3640, a hydrogel plug 3650 disposed between formulation 3640 and plug 3620, a piston 3670, and an expander 3655 disposed between piston 3670 and plug 3625. Elution, and diffusion through orifice 3630, is similar to that described with respect to orifice 3430 in FIG. 34. Expander 3655 is a molded dry (e.g., dehydrated) hydrogel loaded with salt to increase a volume of fluid that expander 3655 can absorb, thus increasing a force that expander 3655 exerts against its surroundings. As formulation 3640 degrades, expander 3655 exerts a force against piston 3670, thus possibly increasing a rate of degradation of formulation 3640 and correspondingly increasing degradation, and increasing the flow of eluted formulation 3640 through orifice 3630.

In one or more embodiments, a membrane is disposed in orifice 3635 of FIG. 36.

Aspects of various embodiments illustrated and described in the present disclosure can be combined. For example, for any shell or delivery device designed in accordance with concepts described in this disclosure: electronics can be included in the shell or delivery device with or without an antenna; formulations can be directly disposed within the shell or delivery device; formulations can be separately constructed in tablet or pill form prior to being disposed within the shell or delivery device, or can be disposed in a container or first delivery device and then the container or first delivery device disposed in a second delivery device; multiple chambers can be used; one or more walls can be used; different materials can be used; and so forth.

As discussed above, materials used to construct a shell can be, or can include, degradable materials. Therefore, in addition to designing plugs, sizes and shapes of orifices, and walls in a manner to implement a desired elution profile, the material of the shell can also be designed to contribute to implementation of the desired elution profile. In a first example, the entire shell is degradable at a designed rate of degradation. In a second example, different portions of the shell are degradable at different designed rates of degradation. In a third example, one or more portions of the shell are non-degradable, and one or more portions of the shell are degradable. By using degradable shells or shell portions, an elution profile can be altered, such as to increase diffusion at a particular time in the elution profile, or to increase diffusion of one formulation and not another. A degradable shell can also be designed to degrade after diffusion is expected to be complete, so that the shell eventually is removed by the body's natural flushing or excreting processes. In one or more embodiments, the shell is absorbable, biodegradable, or bioresorbable.

### Coating

A coating (e.g., coating 1420 in FIG. 14) can be disposed over all of, or a portion of, a delivery device such as a delivery device incorporating any of the features discussed herein, to further define an elution profile.

An embodiment of a coating is a degradable coating. A rate of degradation can be designed for a degradable coating for an expected environment of the target delivery location, such as by selection of chemical composition or thickness of the degradable coating.

An embodiment of a coating is a protective coating, such as a protective coating which protects portions of a delivery device from coming into contact with tissues or fluids (e.g., biological tissue or fluid.) One such protective coating is wax, such as beeswax or other wax. In a first example, a protective coating can be used to cover a shell except where a plug is to be positioned, such that only the plug is exposed to fluid. In a second example, a protective coating can be used to cover a shell and also cover a portion of a plug, such as to focus degradation of the plug for a more uniform degradation across the exposed surface of the plug, or such as to use the same shell/plug design to implement multiple elution profiles by adjusting an amount of plug surface exposed by the coating.

Other examples of coatings (or coating layers) include opacifiers, markers, radiopaque markers, and pigments. In one or more embodiments, a coating is, or includes, an immunosuppressant such as P15 or P15(e), to suppress the “foreign body reaction” immune response of a body to objects introduced into the body (e.g., a delivery device in accordance with an embodiment of the present disclosure).

A coating can be constructed as a single layer or as multiple layers. If multiple layers are used, same or similar materials can be used for the different layers, or one or more layers can be of a different material than others of the layers. In a first example of layers of different materials, a protective coating is provided as an inner layer and exposes a portion of a delivery device, and a degradable coating is provided as an outer layer and covers a portion of the inner protective coating layer including the portion of the delivery device exposed by the inner protective coating layer. In this first example of layers of different materials, the outer degradable coating layer is structured to degrade when exposed to an environment at a target delivery site to expose the portion of the delivery device exposed by the inner protective coating layer. In a second example of layers of different materials, a degradable coating is provided as an inner layer covering a delivery device, and a protective coating is provided as an outer layer of the coating and exposes a portion of the inner degradable coating layer. In this second example of layers of different materials, fluid first reaches and begins to degrade the exposed portion of the inner degradable coating layer and then begins to degrade the inner degradable coating layer from under the outer protective coating layer. In a third example, a hydrophobic outer layer is degradable in certain conditions, and an inner layer adjacent to the outer layer is hydrophilic. In this third example, the outer layer degrades due to exposure to the environment, the inner layer absorbs fluid from the environment after the outer layer is breached, and the inner layer then contributes to degradation of the outer layer to increase a degradation rate of the outer layer after it is breached.

Examples of coatings include: a three-layer coating of PLA-Mg-PLA; a two-layer coating of PLA-PLA; a one-layer coating of Mg; a one-layer coating of PLA; a three-layer coating of Mg-PLA-opacifier, where the opacifier is in the outer layer and can also include coloring; a four-layer coating of PLA-Mg-radiopaque marker-opacifier, where the opacifier is in the outer layer and can also include coloring. Many other examples abound.

One or more dissolution zones can be defined by a coating. As used herein, the term dissolution zone refers to a zone in which degradation is designed to occur. There is a single defined dissolution zone in an embodiment in which a coating is designed for uniform coverage of a delivery device (although manufacturing variances can occur.) When multiple dissolution zones are defined, there is a degradation rate design value for each dissolution zone. Degradation rate design values can be implemented, for example, by using different coatings in different dissolution zones, by using different thicknesses of coating in different dissolution zones, by using different numbers of layers of a coating in different dissolution zones, by using different materials in different layers of a coating in different dissolution zones, by scoring (e.g., cutting lines in) a coating in one or more dissolution zones, by forming holes or rows of holes in a coating in one or more dissolution zones, by another technique, or by a combination of two or more techniques.

In one or more embodiments, a protective coating layer includes wax (e.g., beeswax) covering portions of a delivery device and/or portions of a degradable coating layer to define one or more dissolution zones. The wax does not appreciably degrade for long durations in many portions of the body; accordingly, the wax can be used to define one or more dissolution zones within which degradation of the delivery device or the degradable coating layer occurs, and outside of which degradation is avoided (where the wax is applied.)

It is to be understood that although wax is discussed for use in defining dissolution zones, other materials can be used instead. For example, silicon oil or emulsions of wax (e.g., emulsions with vegetable oil, palm oil, or sunflower oil) can be used to define dissolution zones. Materials used to define dissolution zones can be non-degradable, or can have a lower degradation rate than materials used for exposed portions of the delivery device or the degradable coating layer, as applicable. Further, it is to be understood that different dissolution zones can be defined to provide for different degradation rates.

FIG. 37 illustrates an example of an embodiment of a shell 3710 of a delivery device including a coating having multiple layers. The coating includes an innermost layer 3720, a protective layer 3730, and an outermost layer 3740. In one or more embodiments, the coating can include one or more structural mechanisms that can cause the coating to degrade in a controlled manner. For example, a structure of the coating can include one or more breaks 3750 (e.g., cut lines or pinholes) extending partially through the protective layer 3730 to promote degradation of the coating at particular areas of the coating, and/or one or more control segments 3760 positioned to inhibit, minimize, or prevent degradation of the coating at particular areas of the coating (embodiments illustrated as a control segment 3760a covering an area of the protective layer 3730, a control segment 3760b covering an area of the innermost layer 3720, and a control segment 3760c covering an area of the outermost layer 3740). An example of a material used for a control segment 3760 is a wax.

In one or more embodiments, a coating includes a peptide layer. The peptide (e.g., P15) appears to the body as a naturally-occurring substance in the body (e.g., collagen), and thus the body's natural immunosuppression mechanisms can be avoided, to repress the body's rejection of the delivery device.

Desired characteristics of a coating can be incorporated by design, such as design of thickness of coating(s), relative location of a plug and a coating with respect to each other, selection of one or more layers of a degradable coating each having selected properties, selection of a chemical composition of the degradable coating or layers thereof, position and extent of a protective coating, and many other attributes.

### Examples of Implementations

A delivery device in accordance with the present disclosure can be positioned at a target site using various techniques. For example, for embodiments to be used within a body, a delivery device can be placed subcutaneously or intramuscularly through an incision or by an injection or by transdermal placement (e.g., needles poked transdermally which break off and stay under the skin), can be placed endoscopically, can be delivered in an oral device which travels through a gastrointestinal tract and triggers a mechanism to eject the delivery device from the oral device within the gastrointestinal tract, can be positioned during surgery, and so forth. In embodiments to be used in environments other than the body, a delivery device can be placed by hand or by mechanical device.

In various embodiments, a delivery device is positioned in a dental cavity formed by a root canal procedure or formed by a tooth extraction procedure. The dental cavity is then permanently or temporarily covered (e.g., with gutta-percha and a tooth filling or crown for a root canal procedure, or by bone graft material and/or skin cover for a tooth extraction procedure). In a first example of the dental cavity embodiment, the delivery device includes a coating that begins degrading when exposed to conditions in the dental cavity which indicate an environment friendly to bacterial infection (e.g., pH level below 5.0, or high sulfide concentrations) to combat infection before it occurs or before it progresses, such as by delivering antibiotic or other treatment. In a second example of the dental cavity embodiment, the delivery device includes a coating that begins degrading when exposed to biological matter in the dental cavity, and the rate of degradation of the coating is sufficient to allow completion of either the root canal procedure or the tooth extraction procedure before the delivery device is exposed. In this second example, the delivery device can contain a formulation such as to reduce swelling or to reduce temperature, or a formulation including antibiotics, or a formulation for other treatment.

In various embodiments, a delivery device is positioned in a glioma. A formulation in the delivery device includes a chemotherapeutic agent (e.g., topotecan). For example, the delivery device is structured to provide the chemotherapeutic agent according to a designed elution profile including an initial dose followed by one or more later doses, or an elution profile including a continuous delivery of the chemotherapeutic agent.

In various embodiments, a delivery device is positioned adjacent to or within a cancerous growth. For example, the delivery device is structured to provide a chemotherapeutic agent or a radiotherapeutic agent according to a designed elution profile including an initial dose followed by one or more later doses, or an elution profile including a continuous delivery of the chemotherapeutic agent or radiotherapeutic agent.

In various embodiments, a delivery device is positioned within a body and structured to deliver continuous or periodic doses of a birth control hormone such as progestin in accordance with a designed elution profile.

In various embodiments, a delivery device is positioned within a body and structured to deliver periodic doses of one or more vaccines in accordance with a designed elution profile.

In various embodiments, a delivery device is positioned adjacent to a bladder and structured to deliver a continuous dose or periodic doses of an anticholinergic (e.g., tolterodine tartrate (Detrol LA), oxybutynin chloride (Ditropan), darifenacin (Enablex), mirabegron (Myrbetriq), oxybutynin (Oxytrol), trospium chloride (Sanctura XR), solifenacin (Vesicare)) in accordance with a designed elution profile to relax the bladder and/or to prevent or minimize spasms of the bladder.

In various embodiments, a delivery device is positioned within an infected area and structured to provide treatment (e.g., antifungal treatment or antibiotic treatment) directly to the area in accordance with a designed elution profile.

In various embodiments, a delivery device is positioned at an inflamed site within a body (e.g., a joint, at the ankle, at arthritic areas) and structured to provide an anti-inflammatory agent at the inflamed site in accordance with a designed elution profile, such as to treat joint pain, gout, or arthritis.

In various embodiments, a delivery device is positioned at a surgical site to provide treatment to the surgical site post-surgery in accordance with a designed elution profile, such as to deliver pain relieving medication (e.g., lidocaine) or post-surgical treatment at the site (e.g., antibiotic, antifungal, vasodilator).

In various embodiments, a delivery device is positioned at a site at which an implant has been positioned within the body. The delivery device is structured to provide a peptide adjacent to the implant, such that the peptide surrounds at least a portion of the implant when delivered in accordance with a designed elution profile (multiple delivery devices can be used to increase coverage of the peptide over the implant). The peptide (e.g., P15) appears to the body as a naturally-occurring substance in the body (e.g., collagen), and thus the body's natural immunosuppression mechanisms can be avoided, to repress the body's rejection of the implant.

In various embodiments, a delivery device is positioned within the brain and structured to provide anti-epileptic therapies (e.g., sodium valproate, carbamazepine, lamotrigine, levetiracetam) to a particular site in accordance with a designed elution profile to treat epileptic conditions.

In various embodiments, a delivery device is positioned adjacent to a neuroma and structured to provide an anti-inflammatory agent (e.g., a corticosteroid) to the neuroma in accordance with a designed elution profile.

In various embodiments, a delivery device is positioned in a lung, a pulmonary artery, or a vena cava and structured to deliver an antihypertensive agent (e.g., thiazide diuretic, calcium channel blocker, ACE inhibitor, angiotensin II receptor antagonist (ARBs), beta blocker) in accordance with a designed elution profile, to treat pulmonary hypertension.

In various embodiments, a delivery device is structured to provide a corticosteroid (e.g., benralizumab), and is positioned along the esophagus to reduce a concentration or density of eosinophils along the esophageal wall, or within the lungs to reduce a concentration or density of eosinophils in the lungs.

In various embodiments, a delivery device is positioned behind an eye and structured to provide a treatment for glaucoma in accordance with a designed elution profile (e.g., delivering one or more of an alpha adrenergic agonist such as apraclonidine, brimonidine, epinepherine, or dipivefrin; a beta blocker such as timolol, levobunolol, carteolol, metipranolol, or betatoxol; a carbonic anhydrase inhibitor such as dorzolamide, brinzolamide, etazolamide, or methazolamide; a miotic such as pilocarpine, or echothiophate; a prostaglandin analog such as afluprost ophthalmic solution, latanoprost, bimatoprost, travoprost, unoprostone isopropyl ophthalmic solution, or latanoprostene bunod ophthalmic solution; a rho kinase inhibitor such as netarsudil ophthalmic solution).

In any embodiment, the delivery device can include an osmotic pump such as described above.

Embodiments of the delivery device can provide for local treatments rather than requiring injections or oral delivery. High dosages can be required for injections and oral delivery treatment due to a low bioavailability through such techniques or due to the dosage being absorbed or filtered by other portions of the body (other than the target location), so that an acceptable amount of the dosage can reach the target location. Such high dose treatment can in turn result in high systemic concentrations of the treatment, leading, for example, to cardiac or renal failure. By delivering such treatments directly to the target location, high systemic concentrations can be avoided.

Embodiments of the delivery device can provide for extended delivery periods in accordance with a designed elution profile.

In one or more embodiments, insulin is delivered using a delivery device according to the present disclosure, where the insulin is delivered over hours, days, or weeks. The delivery device is sized to contain a sufficient quantity of insulin for the desired elution profile of the insulin at a target delivery site. For example, the delivery device can be positioned (manually, or through mechanical means such as by way of a mechanism which ejects the delivery device from a container traveling through the gastrointestinal tract) within a wall of the intestinal tract (e.g., stomach wall, intestinal wall) or within the peritoneal cavity to deliver the insulin directly into vascularized portions of a body over several days (e.g., 2-3 days). In this example, the insulin is used to replace the alternative treatment of multiple daily basal insulin injections (e.g., twice per day). Accordingly, because patient compliance can be low with injections, compliance using a delivery device in accordance with the present disclosure is expected to be significantly higher. Moreover, by maintaining basal insulin delivery substantially steadily over many days or weeks, a need for mealtime bolus insulin can be reduced, and such bolus insulin can also be delivered by way of a delivery device in accordance with the present disclosure.

Similarly to the foregoing example of insulin, in one or more embodiments, an incretin mimetic (e.g., exenatide) is delivered using a delivery device according to the present disclosure, where the incretin mimetic is delivered over hours, days, or weeks. For example, repeated four-hour half-life injections of incretin mimetic can be replaced by a single delivery device providing the incretin mimetic substantially steadily over many days or weeks.

Similarly to the foregoing example of insulin, in one or more embodiments, a GLP-1 receptor agonist is delivered using a delivery device according to the present disclosure, where the GLP-1 receptor agonist is delivered over hours, days, or weeks. For example, repeated four-hour half-life injections of GLP-1 receptor agonist can be replaced by a single delivery device providing the GLP-1 receptor agonist substantially steadily over many days or weeks.

Similarly to the foregoing example of insulin, in one or more embodiments, somatostatin or an analog or mimetic thereof is delivered over hours, days, or weeks. For example, repeated five-hour half-life injections of somatostatin or an analog or mimetic thereof can be replaced by a single delivery device providing the somatostatin or an analog or mimetic thereof substantially steadily over many days or weeks.

In sum, a delivery device in accordance with the present disclosure can, in effect, extend a half-life of many therapeutic agents while reducing caregivers' compliance concerns.

Although in many instances herein the various delivery device embodiments and their constituent components have been described as being degraded upon exposure to biological matter in accordance with an elution profile, in other embodiments, degradation is designed to occur under other environmental conditions. In one or more embodiments, an elution profile of a delivery device is designed with respect to exposure to a specific chemical, chemical compound, or combination of chemicals and/or chemical compounds rather than biological matter. For example, microbes can be released after detecting oil, such as to clean up oil spills. For another example, cobalt oxide nanoparticle ligands can be released after detecting carbon monoxide, such as to oxidize the carbon monoxide to carbon dioxide. For a further example, treatment chemicals can be released into a pool after detecting an excess or lack of chlorine. As can be seen by these examples, the techniques of the present disclosure are applicable to a wide variety of environments and technology areas.

Various applications of the delivery device can use multiple delivery devices. Such multiple delivery devices can be similar to each other, or one or more can be different from the others. For example, multiple delivery devices with different agents and/or different elution profiles can be disposed in a body throughout a tumor, surrounding a cancerous tissue, within a lung or other organ, along a nerve, adjacent a joint, along a length within the gastrointestinal tract (e.g., esophagus, stomach, intestine) and so forth. For another example, multiple delivery devices can be disposed into a reservoir, stream, or other water body, or waste water holding tank, to deliver treatment to the water, such as for cleaning or for adding nutrients (e.g., for aquatic life or plants). Other examples abound, such as the treatments for oil spills, carbon monoxide, and chlorine described above.

A delivery device in accordance with the present disclosure provides for a designed elution profile of a formulation from the delivery device. In addition to a delivery device and its constituent components being designed to have respective rates of degradation, a formulation can be formed in a structure and/or include various excipients to achieve a desired rate of degradation when exposed to a fluidic environment. In one or more embodiments, a formulation includes a combination of PLGA and an agent, where the agent is contained within a PLGA matrix and is released when the PLGA matrix degrades. A delivery device can include multiple formulations, one or more of which includes such a PLGA matrix and agent. Matrices other than PLGA matrices can additionally or alternatively be incorporated in a delivery device, for a designed elution profile.

A delivery device in accordance with the present disclosure can be sized in accordance with the intended usage of the delivery device. A delivery device can be sized to accommodate a technique for positioning the delivery device, and/or sized to contain a desired amount of formulation, and/or to meet another design target. For example, if a delivery device is placed surgically, it can have dimensions appropriate for the surgical site, taking into account the effect of the delivery device on its surroundings and space constraints placed on the location due to bone, muscle, cartilage, nerves, vessels, organ walls, and also taking into account a number of delivery devices placed at the surgical site (e.g., around a tumor, or around an implant); accordingly, dimensions of a surgically-placed delivery device can be in terms of millimeters (mm) (e.g., a width, length, or diameter in a range of 1 mm-10 mm, in a range of 5 mm-10 mm, in a range of 10 mm-20 mm, in a range of 20 mm-50 mm, greater than 5 mm, less than 15 mm, less than 35 mm; a circumference of 100 mm or less, in a range of 20 mm-60 mm) or in terms of centimeters (cm) (e.g., a width, length, or diameter less than 10 cm, in a range of 1 cm-1.5 cm, in a range of 1 cm-5 cm, greater than or equal to 1 cm; a circumference of less than 4 cm, in a range of 0.5 cm-1 cm). If a delivery device is provided for oral delivery inside another device (e.g., inside a capsule or inside an automated delivery system within another device), then the delivery device is sized appropriately to fit inside that other device.

Formulation amounts disposed in a delivery device can be determined in accordance with a cavity or chamber size of the delivery device, and/or an amount of formulation to be delivered, and/or other design target. For example, a delivery device configured for automatic deployment of the delivery device out of a swallowed device into a wall of a gastrointestinal tract can contain a formulation amount in terms of milligrams (mg) (e.g., about 1 mg, less than 5 mg, about 8 mg, in a range of 9 mg-10 mg). For another example, a delivery device can be sized to contain large quantities (e.g., in terms of tens or hundreds of grams, or more) of formulation, such as to provide large quantities of the formulation at a target location (e.g., body of water) or to provide the formulation for an extended period of time (e.g., months or years).

In one or more embodiments, a delivery device includes a formulation including a first amount of a therapeutic agent interspersed with a second amount of a delay agent, the formulation having a pre-defined degradation rate. The delivery device further includes a shell encapsulating the formulation and a plug disposed at an orifice defined by the shell.

In one or more embodiments, a device for controlling a delivery profile of a therapeutic substance includes a shell, a plug, and a formulation. The shell defines an orifice extending from an exterior of the shell to an interior of the shell, and the shell further defines a cavity in communication with the orifice. The plug is disposed at the orifice and is structured to prevent fluid from entering the cavity until a predefined condition occurs. The formulation is disposed within the cavity, and the formulation includes the therapeutic substance.

In one or more embodiments, a delivery device includes an osmotic pump, a shell, and a formulation. The osmotic pump includes an expander comprising a dry combination of hydrogel and salt structured to expand in the presence of fluid, and a piston adjacent to the expander and structured to move responsively to force exerted on the piston by expansion of the expander. The shell defines a cavity and further defines two orifices in communication with the cavity. The osmotic pump is disposed within the cavity, the shell being structured to permit fluid to enter the cavity through a first of the two orifices to come into contact with the expander, the shell being further structured to permit fluid to enter the cavity through a second of the two orifices. The formulation is disposed adjacent to the piston in the cavity and structured to degrade in the presence of fluid entering the cavity through the second of the two orifices, thereby forming a fluidized formulation, the shell structured such that movement of the piston forces the fluidized formulation out of the shell through the second of the two orifices.

In one or more embodiments, a method of forming a delivery device includes: providing a shell material having a predefined degradation rate; forming the shell material into a shell defining a cavity and further defining an orifice; disposing into the cavity a formulation; positioning at the orifice a plug structured to block the orifice; and providing the delivery device for ingestion or implantation into a human or other animal.

In any of the foregoing embodiments, the shell can include PGA, PLA, PLGA, or a combination of two or more of the foregoing. In any of the foregoing embodiments, the shell can include magnesium. In any of the foregoing embodiments, the shell can be structured in two or more layers.

In any of the foregoing embodiments, the delivery device can be structured such that a degradation rate of the shell is slower than a degradation rate of the formulation.

In any of the foregoing embodiments, the delivery device can be structured such that a degradation rate of the shell is slower than a degradation rate of a plug of the delivery device. In any of the foregoing embodiments, a plug of the delivery device can include magnesium. In any of the foregoing embodiments, a plug of the delivery device can be structured to have a first portion disposed in an orifice of the shell and a second portion exposed from the orifice. The second portion can include a pointed end. In any of the foregoing embodiments, a plug of the delivery device can be structured to include a degradable metal portion encased by the plug.

In any of the foregoing embodiments, the therapeutic agent can include basal insulin. In any of the foregoing embodiments, the therapeutic agent can include a peptide.

In any of the foregoing embodiments, the formulation can include a delay agent. In any of the foregoing embodiments, a delay agent can include one or both of PGA and PLA. In any of the foregoing embodiments, a delay agent can include PEG, a hydrogel, PEO, or a combination of two or more of the foregoing.

In any of the foregoing embodiments, the delivery device can include a tracking component. The tracking component can be an electronic circuit structured to collect information and wirelessly transmit the collected information to a remote receiver. The tracking component can be a radiopaque substance.

In any of the foregoing embodiments, the delivery device can include a plug disposed at an orifice in the shell and structured to prevent fluid from entering a cavity or chamber of the shell until a predefined condition occurs. The predefined condition can be a predefined threshold or range of time, temperature, or pH. The predefined condition can be a combination of predefined values, and each predefined value is a threshold or a range of time, temperature, or pH.

In any of the foregoing embodiments, the delivery device can include a plug disposed at an orifice in the shell. The plug can be disposed over the orifice, disposed within the orifice, and/or disposed inside a cavity of the shell. In any of the foregoing embodiments, the delivery device can include multiple plugs disposed in a single orifice, or multiple plugs each disposed in separate orifices, or multiple plugs disposed in a single orifice and at least one plug disposed in a separate orifice. In embodiments with multiple plugs, the plugs can be structured to degrade within a same or similar time period, or to degrade at different degradation rates. Accordingly, a first plug can be structured to degrade within minutes of a second plug, or the first plug can be structured to withstand degradation for several minutes, hours, days, weeks, months, or years after the second plug is breached. In embodiments with multiple plugs, the plugs can be structured to have similar shapes, or a plug can have a shape structured differently than one or more other plugs. In any of the foregoing embodiments, the plug can include a hydrogel.

In any of the foregoing embodiments, multiple formulations can be disposed in the delivery device, and each formulation can include one or more agents. In any of the foregoing embodiments, the delivery device can include multiple chambers. In embodiments with multiple chambers, multiple formulations can be disposed in one or more of the multiple chambers: different ones of the multiple chambers can each contain a different one or more formulations; or different ones of the multiple chambers can each contain a same formulation, either in a same volume or dosage or in different volumes or dosages. A chamber can be left empty.

In any of the foregoing embodiments, the delivery device can include electronic circuitry. The electronic circuitry can be structured to detect fluid and, based on detecting the fluid, cause a sample to be collected in a sample collector in the delivery device. The electronic circuitry can be structured to detect fluid and, based on detecting the fluid, cause a biomarker to be disposed external to the delivery device. The electronic circuitry can be structured to detect fluid and, based on detecting the fluid, transmit a message external to the delivery device.

In any of the foregoing embodiments, the delivery device and the formulation can be structured to provide a predefined elution profile for a therapeutic agent as the formulation is diffused from the delivery device under expected environmental conditions.

### CONCLUSION

In sum, a desired elution profile can be identified, and a delivery device according to the present disclosure can be designed to effect the desired elution profile, such as by selecting materials for the constituent components of the delivery device, designing a structure of the delivery device and its constituent components, and/or selecting a content of formulations, to provide for designed degradation (or designed lack of degradation) at particular expected conditions or at particular times or both.

While the present disclosure has been described and illustrated with reference to specific embodiments thereof, these descriptions and illustrations do not limit the present disclosure. It can be clearly understood that various changes can be made, and equivalent components can be substituted within the embodiments without departing from the true spirit and scope of the present disclosure as defined by the appended claims. Also, elements, characteristics, or acts from one embodiment can be readily recombined or substituted with one or more elements, characteristics or acts from other embodiments to form numerous additional embodiments within the scope of the invention. Moreover, elements that are shown or described as being combined with other elements, can, in various embodiments, exist as standalone elements. Further, for any positive recitation of an element, characteristic, constituent, feature, step or the like, embodiments of the invention specifically contemplate the exclusion of that element, value, characteristic, constituent, feature, step or the like. The illustrations may not necessarily be drawn to scale. There can be distinctions between the artistic renditions in the present disclosure and the actual apparatus, due to variables in manufacturing processes and such. There can be other embodiments of the present disclosure which are not specifically illustrated. The specification and drawings are to be regarded as illustrative rather than restrictive. Modifications can be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it can be understood that these operations can be combined, sub-divided, or re-ordered to form an equivalent method without departing from the teachings of the present disclosure. Therefore, unless specifically indicated herein, the order and grouping of the operations are not limitations of the present disclosure.

1. A delivery device, comprising:
a predefined first amount of a therapeutic agent;
a predefined second amount of a delay agent;
a formulation comprising the first amount of the therapeutic agent interspersed with the second amount of the delay agent, the formulation having a pre-defined degradation rate;
a shell encapsulating the formulation, the shell defining a cavity in which the formulation is disposed, the shell further defining an orifice in communication with the cavity; and
a plug disposed at the orifice.

2. The delivery device of claim 1, wherein the shell comprises poly(glycolic acid) (PGA) or poly(lactic acid) (PLA), or a combination of PGA and PLA.

3. The delivery device of claim 1, wherein the shell comprises poly(lactic-co-glycolic acid) (PLGA).

4. The delivery device of claim 1, structured so that a degradation rate of the shell is slower than the degradation rate of the formulation.

5. The delivery device of claim 1, wherein the shell comprises magnesium.

6. The delivery device of claim 1, wherein the shell is structured in two or more layers and at least one of the layers comprises magnesium.

7. The delivery device of claim 1, structured so that a degradation rate of the plug is faster than a degradation rate of the shell.

8. The delivery device of claim 1, wherein the plug comprises magnesium.

9. The delivery device of claim 1, the plug comprising a first portion disposed in the orifice and a second portion exposed from the orifice, wherein the second portion comprises a pointed end.

10. The delivery device of claim 9, further comprising a degradable metal portion encased by the pointed end.

11. The delivery device of claim 1, wherein the therapeutic agent comprises basal insulin.

12. The delivery device of claim 1, wherein the therapeutic agent comprises a peptide.

13. The delivery device of claim 1, wherein the delay agent comprises one of PGA or PLA.

14. The delivery device of claim 1, wherein the delay agent comprises PGA and PLA.

15. The delivery device of claim 1, wherein the delay agent comprises poly(ethylene glycol) (PEG).

16. The delivery device of claim 1, wherein the delay agent comprises a hydrogel and poly(ethylene oxide) (PEO).

17. The delivery device of claim 1, further comprising a tracking component.

18. The delivery device of claim 17, wherein the tracking component is an electronic circuit structured to collect information and wirelessly transmit the collected information to a remote receiver.

19. The delivery device of claim 17, wherein the tracking component is a radiopaque substance.

20. A delivery device for controlling a delivery profile of a therapeutic substance, the delivery device comprising:
a shell defining an orifice extending from an exterior of the shell to an interior of the shell, the shell further defining a cavity in communication with the orifice;
a plug disposed at the orifice and structured to prevent fluid from entering the cavity until a predefined condition occurs; and
a formulation disposed within the cavity, the formulation comprising the therapeutic substance.

21. The delivery device of claim 20, wherein the predefined condition is a predefined threshold or range of time, temperature, or pH.

22. The delivery device of claim 20, wherein the predefined condition is a combination of predefined values, and each predefined value is a threshold or a range of time, temperature, or pH.

23. The delivery device of claim 20, wherein the plug comprises a hydrogel.

24. The delivery device of claim 20, wherein the plug is disposed over the orifice.

25. The delivery device of claim 20, wherein the plug is disposed within the orifice.

26. The delivery device of claim 20, wherein the plug is disposed inside the cavity.

27. The delivery device of claim 20, wherein a portion of the plug is disposed within the orifice and a portion of the plug extends outside of the shell.

28. The delivery device of claim 20, wherein the orifice is a first orifice and the plug is a first plug, and wherein the shell defines two or more orifices including the first orifice, and wherein the delivery device comprises two or more plugs including the first plug.

29. The delivery device of claim 28, wherein the first plug is structured to have a degradation rate greater than the degradation rate of a second plug of the two or more plugs, such that the first plug is structured to degrade at least one hour faster than the second plug is structured to degrade.

30. The delivery device of claim 28, wherein the first plug is structured to have a degradation rate approximately equal to the degradation rate of a second plug of the two or more plugs, such that the first plug and the second plug degrade within minutes of each other.

31. The delivery device of claim 30, wherein the first plug and the second plug are structured to have different shapes.

32. The delivery device of claim 20, further comprising multiple chambers, wherein the cavity is in communication with at least one of the multiple chambers.

33. The delivery device of claim 32, wherein the formulation is a first therapeutic formulation, further comprising a plurality of therapeutic formulations including the first therapeutic formulation, and wherein each of the multiple chambers contains a different one or more of the plurality of therapeutic formulations.

34. The delivery device of claim 32, wherein each of the chambers contains a volume of the formulation.

35. The delivery device of claim 20, further comprising a sample collector and electronic circuitry, wherein the electronic circuitry is structured to detect fluid and, based on detecting the fluid, cause a sample to be collected in the sample collector.

36. The delivery device of claim 20, further comprising electronic circuitry structured to detect fluid and, based on detecting the fluid, cause a biomarker to be disposed external to the delivery device.

37. The delivery device of claim 20, further comprising electronic circuitry structured to detect fluid and, based on detecting the fluid, transmit a message external to the delivery device.

38. The delivery device of claim 20, wherein the delivery device and the formulation are structured to provide a predefined elution profile for the therapeutic substance as it is diffused from the delivery device under expected environmental conditions.

39. A delivery device comprising:
an osmotic pump, the osmotic pump comprising:
an expander comprising a dry combination of hydrogel and salt structured to expand in the presence of a fluid;
a piston adjacent to the expander and structured to move responsively to force exerted on the piston by expansion of the expander;
a shell defining a cavity and further defining two orifices in communication with the cavity, the osmotic pump disposed within the cavity, the shell being structured to permit fluid to enter the cavity through a first of the two orifices to come into contact with the expander, the shell being further structured to permit fluid to enter the cavity through a second of the two orifices; and
a formulation disposed adjacent to the piston in the cavity and structured to degrade in the presence of fluid entering the cavity through the second of the two orifices, thereby forming a fluidized formulation, the shell structured such that movement of the piston will force the fluidized formulation out of the shell through the second of the two orifices.

40. A method of forming a delivery device, the method comprising:
providing a shell material having a predefined degradation rate;
forming the shell material into a shell defining a cavity and further defining an orifice;
disposing into the cavity a formulation;
positioning at the orifice a plug structured to block the orifice; and
providing the delivery device for ingestion or implantation into a human or other animal.
